(19) United States
(12) Patent Application Publication
(10) Pub. No.: US 2017/0233727 A1
(43) Pub. Date: Aug. 17, 2017

(54) METHODS FOR GENERATING AND DECODING BARCODES

(71) Applicants: Wei ZHOU, Saratoga, CA (US); CENTRILLION TECHNOLOGY HOLDING CORPORATION, Grand Cayman, KY (US)

(72) Inventors: Wei ZHOU, Saratoga, CA (US); Scott T. POLLOM, Menlo Park, CA (US)

(73) Assignee: CENTRILLION TECHNOLOGY HOLDINGS CORPORATION, Grand Cayman, KY (US)

(21) Appl. No.: 15/309,941

(22) PCT Filed: May 20, 2015
(86) PCT No.: PCT/US2015/031732

§ 371 Date: Nov. 9, 2016

Related U.S. Application Data
(60) Provisional application No. 62/002,759, filed on May 23, 2014.
(60) Provisional application No. 62/064,945, filed on Oct. 16, 2014.

(51) Int. Cl. C12N 15/10

(52) U.S. Cl. CPC C12N 15/1093

(57) ABSTRACT

The present disclosure provides methods and systems for generating and decoding a set of barcodes, which include the utilization of a hash function. The disclosure also related to kits that are suitable for carrying out the inventive methods.

### CROSS-REFERENCE

This application claims priority to U.S. Provisional Patent Application Ser. No. 62/002,759, filed May 23, 2014, and U.S. Provisional Patent Application Ser. No. 62/064,945, filed Oct. 16, 2014, each of which is incorporated herein by reference in its entirety.

### BACKGROUND

Barcodes permit faster and more accurate recording of information. Matching can move quickly and be tracked precisely with the use of barcodes. Quite a bit of time can be spent tracking down the location or status of target substances such as samples, projects, folders, instruments, and materials. Better barcode design can help to greatly save time and reduce errors.

Barcoding and barcode design can be applicable to a variety of contexts, such as sample processing, analysis and sequencing. Advances in DNA sequencing have resulted in instruments of remarkable performance, including extraordinary base read rates, and enormous sequencing depths. Sample throughput, nevertheless, remains slow, a situation that could be alleviated through sample multiplexing, with the incorporation of oligonucleotide tags or barcodes serving to identify the different samples. The quality of the resulting sequence data is directly impacted by the quality of the barcodes. Methods for high-quality barcode design are needed in advanced sequencing applications.

### SUMMARY

The throughput of next generation sequencing technology has increased rapidly over the past 10 years. Due to the large increases in sequencing capacity, a growing need for massive numbers of oligonucleotide sequence identification tags (DNA barcodes) has emerged. DNA barcodes can be attached to individual strands of DNA during library preparation before sequencing in order to determine the source of each read after sequencing. The increasing throughput of next-generation DNA sequencing may create new opportunities to utilize large sets of DNA barcodes; e.g., a large set of DNA barcodes may be necessary to perform low-coverage sequencing on a large set of samples in parallel.

When designing a set of DNA barcodes, requiring a minimum number of substitutions, insertions, or deletions (or edit distance) to convert one barcode into another may be of great importance, because if two barcodes in the set are too similar, then one can be mistaken for the other if errors occur during synthesis, amplification, or sequencing.

The present disclosure provides methods and systems for generating a set of barcodes and decoding a set of potentially changed barcodes.

An aspect of the present disclosure provides a set of barcodes comprising at least 1,500,000 barcodes with an edit distance of at least 2. In some embodiments of aspects provided herein, the set of barcodes comprises at least 5,000,000 barcodes. In some embodiments of aspects provided herein, the set of barcodes comprises at least 10,000,000 barcodes. In some embodiments of aspects provided herein, the edit distance is at least 4. In some embodiments of aspects provided herein, each of the barcodes has a length of at least 10. In some embodiments of aspects provided herein, each of the barcodes has a length of at least 15. In some embodiments of aspects provided herein, the set of barcodes has an error rate of 0.005% or less. In some embodiments of aspects provided herein, the set of barcodes has an error rate of 0.001% or less. In some embodiments of aspects provided herein, the barcodes comprise nucleic acid molecules. In some embodiments of aspects provided herein, additional information is associated with the barcodes. In some embodiments of aspects provided herein, the additional information comprises at least one of: (a) a complete nucleic acid sequence; (b) a source identifier; and (c) an information link. In some embodiments of aspects provided herein, the barcodes have a G:C content above a pre-determined threshold value. In some embodiments of aspects provided herein, the barcodes have a G:C content below a pre-determined threshold value. In some embodiments of aspects provided herein, the barcodes have less than four nucleotides in a row from the group consisting of A and T. In some embodiments of aspects provided herein, the barcodes have less than four nucleotides in a row from the group consisting of G and C. In some embodiments of aspects provided herein, the barcodes have a homopolymer run less than or equal to 4 nucleotides in length.

Another aspect of the present disclosure provides a method for generating a set of barcodes having a pre-determined library edit distance, comprising: (a) providing a set of library barcodes, wherein each of the library barcodes in the set of library barcodes comprises a library barcode index; (b) receiving a candidate barcode; (c) generating a first set of mutations of the candidate barcode; (d) converting the candidate barcode, each of the library barcodes and each of the first set of mutations of the candidate barcode into hash values using a hash function; (e) providing a creation hash table that relates each of the hash values of each of the library barcodes to its library barcode index; (f) comparing the hash values of the first set of mutations of the candidate barcode to the creation hash table, and if at least one of the hash values has been assigned to the library barcode index or indices in the creation hash table, then determining edit distances between the candidate barcode and the library barcode or the library barcodes indexed with the same hash value; and (g) adding the candidate barcode to the set of library barcodes if none of the determined edit distances from step (f) are less than the pre-determined library edit distance.

In some embodiments of aspects provided herein, the set of library barcodes is empty and the candidate barcode is added to the set of library barcodes without comparison. In some embodiments of aspects provided herein, the set of library barcodes comprises at least one library barcode. In some embodiments of aspects provided herein, the creation hash table is empty. In some embodiments of aspects provided herein, each of the library barcodes has a length of at least 2. In some embodiments of aspects provided herein, each of the library barcodes has a length of at least 10. In some embodiments of aspects provided herein, the candidate barcode has a length of at least 2. In some embodiments of aspects provided herein, the candidate barcode has a length of at least 10. In some embodiments of aspects provided herein, the library edit distance is at least 2. In some embodiments of aspects provided herein, the library edit distance is at least 4. In some embodiments of aspects provided herein, the method further comprises determining a comparison edit distance according to the library edit distance. In some embodiments of aspects provided herein, the comparison edit distance is determined by using the formula [the library edit distance−1−integer ((the library edit distance−1)/2)]. In some embodiments of aspects provided herein, the comparison edit distance is 0. In some embodiments of aspects provided herein, the comparison edit distance is at least 1. In some embodiments of aspects provided herein, the method further comprises determining a creation hash table edit distance according to the library edit distance. In some embodiments of aspects provided herein, the creation hash table edit distance is determined by using the formula [integer ((the library edit distance−1)/2)]. In some embodiments of aspects provided herein, the creation hash table edit distance is 0. In some embodiments of aspects provided herein, the creation hash table edit distance is at least 1. In some embodiments of aspects provided herein, the method further comprises: determining a creation hash table edit distance and a comparison edit distance according to the library edit distance by using the formula [the library edit distance=the creation hash table edit distance+the comparison edit distance+1]. In some embodiments of aspects provided herein, the first set of mutations of the candidate barcode is within the comparison edit distance of the candidate barcode. In some embodiments of aspects provided herein, the method further comprises: (i) generating one or more mutations of at least one of the library barcodes, wherein the mutations are within the creation hash table edit distance of the at least one of the library barcodes; (ii) converting the one or more mutations from (i) into hash values using the hash function; and (iii) relating the hash values from (ii) to the library barcode index of the at least one of the library barcode in the creation hash table. In some embodiments of aspects provided herein, the method further comprises: (h) assigning a new library barcode index to the added candidate barcode; (i) generating a second set of mutations of the added candidate barcode, wherein the second set of mutations is within the creation hash table edit distance of the added candidate barcode; (j) determining hash values of the second set of mutations of the added candidate barcode using the hash function; and (k) updating the creation hash table by pairing the new library barcode index with the hash values of the second set of mutations of the added candidate barcode. In some embodiments of aspects provided herein, the method further comprises receiving a set of candidate barcodes and selecting an individual candidate barcode from the set of candidate barcodes. In some embodiments of aspects provided herein, the individual candidate barcode is selected in a random order. In some embodiments of aspects provided herein, the individual candidate barcode is selected in an order. In some embodiments of aspects provided herein, the method further comprises selecting the next candidate barcode from the set of candidate barcodes if none of the hash values of the first set of mutations of the selected candidate barcode have been assigned to the library barcode index in the creation hash table. In some embodiments of aspects provided herein, the method further comprises keeping selecting the candidate barcode for comparison until the set of library barcodes comprises a pre-determined number of barcodes. In some embodiments of aspects provided herein, the set of library barcodes comprises a plurality of nucleic acid molecules. In some embodiments of aspects provided herein, the set of library barcodes is contained in a file. In some embodiments of aspects provided herein, the set of candidate barcodes comprises a plurality of nucleic acid molecules. In some embodiments of aspects provided herein, the set of candidate barcodes is contained in a file. In some embodiments of aspects provided herein, the method further comprises removing the candidate barcode with a G:C content above a pre-determined threshold value. In some embodiments of aspects provided herein, the method further comprises removing the candidate barcode with a G:C content below a pre-determined threshold value. In some embodiments of aspects provided herein, the method further comprises removing the candidate barcode capable of forming a hairpin structure. In some embodiments of aspects provided herein, the method further comprises removing the candidate barcode having a known restriction site. In some embodiments of aspects provided herein, the method further comprises removing the candidate barcode having a start codon. In some embodiments of aspects provided herein, the method further comprises removing the candidate barcode having forbidden sequences. In some embodiments of aspects provided herein, the method further comprises removing the candidate barcode having more than three nucleotides in a row from the group consisting of A and T. In some embodiments of aspects provided herein, the method further comprises removing the candidate barcode having more than three nucleotides in a row from the group consisting of G and C. In some embodiments of aspects provided herein, the method further comprises removing the candidate barcode having a homopolymer run greater than or equal to 2 nucleotides in length. In some embodiments of aspects provided herein, the method further comprises removing the candidate barcode having a homopolymer run greater than or equal to 4 nucleotides in length. In some embodiments of aspects provided herein, the method further comprises removing the candidate barcode that is complementary to an mRNA sequence in an organism. In some embodiments of aspects provided herein, the method further comprises removing the candidate barcode that is complementary to a genomic sequence in an organism. In some embodiments of aspects provided herein, the method further comprises removing the candidate barcode having a melt temperature below a pre-determined threshold value. In some embodiments of aspects provided herein, the method further comprises removing the candidate barcode having a melt temperature above a pre-determined threshold value.

In some embodiments of aspects provided herein, the set of barcodes comprises at least 10,000 barcodes. In some embodiments of aspects provided herein, the set of barcodes comprises at least 100,000 barcodes. In some embodiments of aspects provided herein, the set of barcodes comprises at least 1,000,000 barcodes. In some embodiments of aspects provided herein, the set of barcodes comprises at least 10,000,000 barcodes. In some embodiments of aspects provided herein, the set of barcodes is generated in less than 500 hours. In some embodiments of aspects provided herein, the set of barcodes is generated in less than 250 hours. In some embodiments of aspects provided herein, the set of barcodes is generated in less than 100 hours. In some embodiments of aspects provided herein, the set of barcodes is generated in less than 50 hours. In some embodiments of aspects provided herein, the set of barcodes is generated with a unit execution time of 1 s or less. In some embodiments of aspects provided herein, the set of barcodes is generated with a unit execution time of 0.1 s or less. In some embodiments of aspects provided herein, the set of barcodes is generated with a unit execution time of 0.01 s or less. In some embodiments of aspects provided herein, the set of barcodes is generated with a unit execution time of 0.001 s or less. In some embodiments of aspects provided herein, the set of barcodes is used for nucleic acid sequencing.

Another aspect of the present disclosure provides a method for decoding a set of barcodes within a pre-determined resolution edit distance, the method comprising: (a) providing a set of library barcodes with the resolution edit distance, wherein each of the library barcodes in the set of library barcodes has a library barcode index; (b) selecting a candidate barcode from the set of barcodes; (c) converting the candidate barcode and each of the library barcodes into hash values using a hash function; (d) providing a decoding hash table that relates each of the hash values of the library barcodes to its library barcode index; (e) comparing the hash value of the candidate barcode to the decoding hash table, and if the hash value of the candidate barcode has already been assigned to the library barcode index or indices in the decoding hash table, then determining edit distances between the candidate barcode and the library barcode or the library barcodes indexed with the same hash value; and (f) matching the candidate barcode to the library barcode or library barcodes if the determined edit distances from step (e) are not greater than the resolution edit distance.

In some embodiments of aspects provided herein, the set of library barcodes is empty and the candidate barcode is added to the set of library barcode without comparison. In some embodiments of aspects provided herein, the resolution edit distance is at least 1. In some embodiments of aspects provided herein, the resolution edit distance is at least 4. In some embodiments of aspects provided herein, each of the library barcodes has a length of at least 2. In some embodiments of aspects provided herein, each of the library barcodes has a length of at least 10. In some embodiments of aspects provided herein, the candidate barcode has a length of at least 2. In some embodiments of aspects provided herein, the candidate barcode has a length of at least 10. In some embodiments of aspects provided herein, the candidate barcode has the same length as the library barcodes. In some embodiments of aspects provided herein, the candidate barcode has a different length as the library barcodes. In some embodiments of aspects provided herein, the method further comprises: (i) generating one or more mutations of at least one of the library barcodes, wherein the one or more mutations are within the resolution edit distance of the at least one of the library barcodes; (ii) converting each of the mutations of the at least one of the library barcodes into hash values using the hash function; and (iii) relating the hash values of the mutations of the at least one of the library barcodes to its library barcode index in the decoding hash table. In some embodiments of aspects provided herein, the candidate barcode is selected from the set of barcodes in a random order. In some embodiments of aspects provided herein, the candidate barcode is selected from the set of barcodes in an order. In some embodiments of aspects provided herein, the method further comprises marking the candidate barcode as “unresolvable” if all of the determined edit distances from step (e) are greater than the resolution edit distance. In some embodiments of aspects provided herein, the method further comprises repeating steps (b)-(f) until a pre-determined number of the candidate barcodes has been decoded. In some embodiments of aspects provided herein, the set of library barcodes comprises nucleic acid molecules. In some embodiments of aspects provided herein, the candidate barcode comprises nucleic acid molecule. In some embodiments of aspects provided herein, the set of barcodes comprises at least 100,000 barcodes. In some embodiments of aspects provided herein, the set of barcodes comprises at least 1,000,000 barcodes. In some embodiments of aspects provided herein, the set of barcodes comprises at least 10,000,000 barcodes. In some embodiments of aspects provided herein, the set of barcodes comprises at least 50,000,000 barcodes. In some embodiments of aspects provided herein, the set of barcodes is decoded in less than 1 hour. In some embodiments of aspects provided herein, the set of barcodes is decoded in less than 1,000 seconds. In some embodiments of aspects provided herein, the set of barcodes is decoded in less than 500 seconds. In some embodiments of aspects provided herein, the set of barcodes is decoded in less than 10 seconds. In some embodiments of aspects provided herein, the set of barcodes is decoded with a unit execution time of 0.001 s or less. In some embodiments of aspects provided herein, the set of barcodes is decoded with a unit execution time of 0.0001 s or less. In some embodiments of aspects provided herein, the set of barcodes is decoded with a unit execution time of 0.00001 s or less. In some embodiments of aspects provided herein, the set of barcodes is decoded with a unit execution time of 0.000001 s or less. In some embodiments of aspects provided herein, the set of barcodes is decoded with a determination error rate of 0.1% or less. In some embodiments of aspects provided herein, the set of barcodes is decoded with a determination error rate of 0.01% or less. In some embodiments of aspects provided herein, the set of barcodes is decoded with a determination error rate of 0.001% or less.

Another aspect of the present disclosure provides a computer readable medium comprising codes that, upon execution by one or more computer processors, implements a method for generating a set of barcodes comprising at least 1,500,000 barcodes with a library edit distance of at least 2, in less than 24 hours.

In some embodiments of aspects provided herein, the method comprises: (a) providing a set of library barcodes, wherein each of the library barcodes in the set of library barcodes comprises a library barcode index; (b) receiving a candidate barcode; (c) generating a first set of mutations of the candidate barcode; (d) converting the candidate barcode, each of the library barcodes and each of the first set of mutations of the candidate barcode into hash values using a hash function; (e) providing a creation hash table that relates each of the hash values of each of the library barcodes to its library barcode index; (f) comparing the hash values of the first set of mutations of the candidate barcode to the creation hash table, and if at least one of the hash values has been assigned to the library barcode index or indices in the creation hash table, then determining edit distances between the candidate barcode and the library barcode or the library barcodes indexed with the same hash value; and (g) adding the candidate barcode to the set of library barcodes if none of the determined edit distances from step (f) are less than the pre-determined library edit distance. In some embodiments of aspects provided herein, the method further comprises: determining a creation hash table edit distance and a comparison edit distance according to the library edit distance. In some embodiments of aspects provided herein, the method further comprises: (i) generating one or more mutations of at least one of the library barcodes, wherein the mutations are within the creation hash table edit distance of the at least one of the library barcodes; (ii) converting the one or more mutations from (i) into hash values using the hash function; and (iii) relating the hash values from (ii) to the library barcode index of the at least one of the library barcode in the creation hash table. In some embodiments of aspects provided herein, the method further comprises: (h) assigning a new library barcode index to the added candidate barcode; (i) generating a second set of mutations of the added candidate barcode, wherein the second set of mutations is within the creation hash table edit distance of the added candidate barcode; (j) determining hash values of the second set of mutations of the added candidate barcode using the hash function; and (k) updating the creation hash table by pairing the new library barcode index with the hash values of the second set of mutations of the added candidate barcode. In some embodiments of aspects provided herein, the method further comprises receiving a set of candidate barcodes and selecting an individual candidate barcode from the set of candidate barcodes. In some embodiments of aspects provided herein, the method further comprises selecting the next candidate barcode from the set of candidate barcodes if none of the hash values of the first set of mutations of the selected candidate barcode have been assigned to the library barcode index in the creation hash table. In some embodiments of aspects provided herein, the method further comprises keeping selecting the candidate barcode for comparison until the set of library barcodes comprises a pre-determined number of barcodes. In some embodiments of aspects provided herein, the set of barcodes is generated in less than 10 hours. In some embodiments of aspects provided herein, the set of barcodes is generated in less than 5 hours. In some embodiments of aspects provided herein, the set of barcodes is generated with a unit execution time of 1 s or less.

Another aspect of the present disclosure provides a computer readable medium comprising codes that, upon execution by one or more computer processors, implements a method for decoding a set of barcodes comprising at least 1,500,000 barcodes with a resolution edit distance of at least 1, in less than 1,000 s.

In some embodiments of aspects provided herein, the method comprises: (a) providing a set of library barcodes with the resolution edit distance, wherein each of the library barcodes has a library barcode index; (b) selecting a candidate barcode from the set of barcodes; (c) converting the candidate barcode and each of the library barcodes into hash values using a hash function; (d) providing a decoding hash table that relates each of the hash values of each of the library barcodes to its barcode index; (e) comparing the hash value of the candidate barcode to the decoding hash table, and if the hash value of the candidate barcode has already been assigned to the library barcode index or indices in the decoding hash table, then determining an edit distance between the candidate barcode and the library barcode or the library barcodes indexed with the same hash value; and (f) matching the candidate barcode to the library barcode or library barcodes if the determined edit distance from step (e) is not greater than the resolution edit distance. In some embodiments of aspects provided herein, the method further comprises: (i) generating one or more mutations of at least one of the library barcodes; (ii) converting the one or more mutations of the at least one of the library barcodes into hash values using the hash function; and (iii) relating the hash values of the one or more mutations of the at least one of the library barcodes to its library barcode index in the decoding hash table. In some embodiments of aspects provided herein, the method further comprises marking the candidate barcode as “unresolvable” if all of the determined edit distances from step (e) are greater than the resolution edit distance. In some embodiments of aspects provided herein, the method further comprises repeating steps (b)-(f) until a pre-determined number of the candidate barcodes has been decoded. In some embodiments of aspects provided herein, the set of barcodes is decoded in less than 300 s. In some embodiments of aspects provided herein, the set of barcodes is decoded in less than 50 s. In some embodiments of aspects provided herein, the set of barcodes is decoded with a unit execution time of 0.000001 s or less. In some embodiments of aspects provided herein, the set of barcodes is decoded with a determination error rate of 1% or less.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary procedure for generating a set of barcodes.

FIG. 2 illustrates an exemplary procedure for decoding a set of barcodes.

FIG. 3 shows the diagram of an exemplary method for generating a set of barcodes.

FIG. 4 shows the diagram of an exemplary method for decoding a set of barcodes.

FIG. 5 shows an exemplary method for generating a set of barcodes.

FIG. 6 shows an example of checking the minimum pairwise edit distance for new barcodes.

FIG. 7 shows execution time of different methods for generating sets of barcodes.

FIG. 8 shows execution time of different methods for decoding sets of barcodes.

FIG. 9 shows sets of barcodes outputted by different methods.

FIG. 10 shows the execution time of different methods for generating sets of barcodes.

### DETAILED DESCRIPTION

### Definitions

As used herein, the singular form “a”, “an”, and “the” include plural references unless the context clearly dictates otherwise.

As used herein, the term “about” refers to the indicated numerical value ±10%.

As used herein, open terms, for example, “contain”, “include”, “including”, and the like refer to comprising unless otherwise indicates.

As used herein, the term “index” refers to a letter, number, symbol, or other representation that uniquely designates a barcode's position within a set of barcodes.

As used herein, the term “hash function” refers to a mathematical manipulation that translates a barcode into a hash value (e.g., whole numbers).

As used herein, the term “hash value” refers to the output of a hash function, which displays a barcode's value after hash function translation.

As used herein, the term “hash table” refers to a plurality of hash values each associated with an index or indices of barcodes.

As used herein, the term “creation hash table” refers to a hash table generated and updated in the method for generating a set of barcodes.

As used herein, the term “decoding hash table” refers to a hash table generated and updated in the method for decoding a set of barcodes.

As used herein, the term “barcode” refers to a sequence of letters, numbers, symbols, or other representations that is distinguishable from other such sequences.

As used herein, the term “edit” refers to any substitution, insertion, or deletion of one letter, number, symbol or other representation in a barcode.

As used herein, the term “edit distance” refers to the minimum number of edits it would take to transform one barcode into another barcode.

As used herein, the term “candidate barcode” refers to a barcode that needs to be decoded, or a barcode that needs to be verified for edit distance requirements before becoming a library barcode.

As used herein, the term “library barcode” refers to a barcode that has passed or would pass the edit distance requirements after the completion of library construction.

As used herein, the term “library edit distance” refers to the minimum number of edits it would take to transform one library barcode into another library barcode, a minimum for which a candidate barcode would need to meet before being accepted by the set of library barcodes.

As used herein, the term “set of library barcodes” refers to a plurality of library barcodes each with an index and different from each other by a specified library edit distance.

As used herein, the term “comparison edit distance” refers to the upper limit of the minimum number of edits it would take to transform a candidate barcode into its mutations.

As used herein, the term “creation hash table edit distance” refers to the upper limit for which the edit distance between a barcode and a library barcode cannot exceed before linking the hash value of the barcode to the index of the library barcode in the creation hash table.

As used herein, the term “resolution edit distance” refers to the minimum number of edits it would take to transform one library barcode into its mutations, and a threshold for which the edit distance between a barcode to be decoded and a corresponding library barcode cannot exceed before matching the barcode to be decoded to the corresponding library barcode.

As used herein, the term “mutation” refers to barcodes that are transformed by a number of edits.

As used herein, the term “error rate” refers to the rate at which a barcode is incorrectly identified as a different barcode.

### General Overview

Provided in the present disclosure are methods and systems for generating and decoding a set of barcodes. Exemplary barcode set generated by methods disclosed herein may comprise at least 1,000,000 n-mer barcodes with an edit distance of 2. Exemplary barcode set decoded by methods disclosed herein may comprise at least 1,000,000 barcodes determined to be within a specified edit distance (e.g., 1, 2, or 4).

In general, a method for generating a set of barcodes having a pre-determined library edit distance may comprise the steps of: (a) providing a set of library barcodes and each of the library barcodes may have a library barcode index: (b) receiving a candidate barcode and generating all possible mutations of the candidate barcode such that each of the mutations is within a creation hash table edit distance of the candidate barcode; (c) converting the candidate barcode, the mutations of the candidate barcode and the library barcodes into hash values by using a hash function; (d) creating a creation hash table and pairing each of the hash values of the library barcodes with its library barcode index in the creation hash table; (e) comparing the hash values of the mutations of the candidate barcode to the creation hash table, and if at least one of the hash values of the mutations of the candidate barcode has already been assigned to one or more of the library barcode indices in the creation hash table, then determining edit distances between the candidate barcode and the library barcode or the library barcodes indexed with the same hash value; and (f) updating the set of library barcodes by adding the candidate barcode to the set of library barcode if none of the determined edit distances from step (e) are less than the library edit distance. In some cases, the method further comprises the steps of: (i) generating one or more mutations of at least one of the library barcodes such that each of the mutations is within a creation hash table edit distance of the library barcode; (ii) calculating hash values of the mutations generated from (i) by using the hash function; and (iii) pairing the calculated hash values from (ii) with the library barcode index of the at least one of the library barcode against which the one or more mutations are generated in the creation hash table.

In some cases, once the candidate barcode has been added to the set of library barcodes and accepted as a new library barcode, a new library barcode index is assigned to the newly added candidate barcode and one or more mutations of the new library barcode are generated such that each of the mutations is within the creation hash table edit distance of the new library barcode. Hash values of these generated mutations may subsequently calculated by using the hash function as disclosed above and elsewhere herein. The hash values of the new library barcode may then be paired with the new library barcode index in the creation hash table.

In some cases, the method further comprises receiving a set of candidate barcodes and selecting an individual candidate barcode for comparison. As discussed elsewhere herein, the individual candidate can be selected randomly or in an order. If after comparison, there is at least one of the determined edit distances from step (e) being less than the library edit distance, then the next candidate barcode is selected from the set of candidate barcodes for comparison. In some cases, the method further comprises keeping selecting the candidate barcode for comparison until a pre-determined number of barcodes have been generated (or repeating steps (b)-(f) until the updated set of library barcodes includes a pre-determined number of barcodes).

Also provided herein are methods for decoding a set of error-correcting barcodes, or barcodes to be decoded. In general, such method may comprise the steps of: (a) providing a set of library barcodes with a pre-determined resolution edit distance; (b) receiving a set of candidate barcodes that need to be decoded and selecting an individual candidate barcode from the set; (c) calculating hash values of the candidate barcode and the library barcodes by using a hash function; (d) creating a decoding hash table and relating each of the hash values of the library barcodes to the corresponding library barcode index in the decoding hash table; (e) comparing the hash value of the candidate barcode to the decoding hash table, and if the hash value has already been assigned to one or more of the library barcode index or indices in the decoding hash table, then determining edit distances between the candidate barcode and the corresponding library barcode or library barcodes indexed with the same hash value; and (f) matching the candidate barcode to the corresponding library barcode or barcodes if the determined edit distances from (e) are not greater than the resolution edit distance. Or, in cases where all of the edit distances from (e) are greater than the resolution distance, then marking the candidate barcode as “unresolvable”.

In some cases, the methods may further comprises steps of: (i) generating one or more mutations of at least one of the library barcodes; (ii) calculating hash values of the generated mutations from (i) by using the hash function as described above and elsewhere herein; and (iii) relating the hash values of the mutations calculated from (ii) to the corresponding library barcode index of the at least one of the library barcode against which the one or more mutations are generated. As discussed above and elsewhere herein, the candidate barcode can be selected randomly or in an order, and the methods may comprise the step of keeping selecting the candidate barcode for comparison until a pre-determined number of barcodes have been decoded.

As provided herein, systems for generating a set of barcodes with a pre-determined edit distance may comprise: (a) a storage unit for storing a creation hash table, a first dataset and a second dataset, wherein the first dataset comprises a plurality of library barcodes and their mutations with a pre-determined library edit distance, and wherein the second dataset comprises a plurality of candidate barcodes and a first set of mutations for each of the candidate barcodes, wherein each of the library barcodes has a library barcode index; (b) a converting unit for converting each of the library barcodes and their mutations, the candidate barcodes and their first set of mutations in the first and the second datasets into a hash value by using a hash function; (c) a first processing unit for assigning each of the converted hash values for the library barcodes and their mutations to the library barcode indices in the creation hash table; (d) a second processing unit for (i) comparing each of the hash values of the first set of mutations of a selected candidate barcodes to the creation hash table; (ii) determining edit distances between the selected candidate barcode and the library barcode or the library barcodes indexed with the same hash value, if at least one of the hash values of its first set of mutations has been assigned to the library barcode index or indices in the creation hash table; (iii) updating the first and the second datasets by adding the selected candidate barcode into the first dataset if none of the determined edit distances between the selected candidate barcode and the corresponding library barcodes are less than the pre-determined library edit distance; and (iv) assigning a new library barcode index to the accepted candidate barcode and generating a second set of mutations for the accepted candidate barcode; (e) a second converting unit for converting each of the second set of mutations for the accepted candidate barcode into a hash value by using the hash function provided in step (b), and linking the resulting hash values with the new library barcode in the creation hash table; and (e) a saving unit for saving the updated creation hash table, and the first and second datasets to a file.

In another example, a system for decoding a set of barcodes is provided, and the system may comprise: (a) a storage unit for storing a first dataset and a second dataset, wherein the first dataset comprises a plurality of library barcodes and mutations of the library barcodes with a pre-determined resolution edit distance, and the second dataset comprises a plurality of barcodes to be decoded, wherein each of the library barcodes has a library barcode index; (b) a converting unit for converting each of the library barcodes, the mutations of the library barcodes and the barcodes to be decoded in the first and the second datasets into a hash value by using a hash function; (c) a first processing unit for assigning each of the converted hash value for the library barcodes and their mutations to the library barcode indices in a decoding hash table; (d) a second processing unit for (i) comparing the hash value of a selected barcode to be decoded to the decoding hash table; (ii) determining an edit distance between the selected barcode to be decoded and the library barcode or the library barcodes indexed with the same hash value, if the hash value of the selected barcode to be decoded has been assigned to the library barcode index or indices in the decoding hash table; and (iii) updating the second datasets by either marking the selected barcode to be decoded as “unresolvable” in the second dataset if all of the determined edit distances are greater than the pre-determined resolution edit distance, or matching the selected barcode to be decoded to one of the corresponding library barcodes if the determined edit distance is not greater than the pre-determined resolution edit distance; and (e) a saving unit for saving the updated second datasets to a file.

Furthermore, the present disclosure provides computer-readable storage media that are capable to implement methods for generating and decoding a set of barcodes. For example, an exemplary computer-readable storage medium may comprise program codes that, upon execution by one or more processors, may implement a method for generating a set of barcodes. In another example, the disclosure provides a computer-readable storage medium that may implement a method for decoding a set of barcodes to be decoded upon the execution of program codes by one or more processors.

Methods, barcode sets, systems and computer-readable media disclosed in the present disclosure may find useful in a wide array of fields and applications. Non-limiting examples of applications may include protein sequencing, nucleotide sequencing, sequencing optimization, optimized barcode design, cataloging, product indexing, security access keys and software purchase keys. In some cases, the present disclosure may provide a faster and more efficient way to generate a large quantity of barcodes with a pre-determined edit distance. Barcode sets generated by the methods of the present disclosure may comprise at least 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000 or more barcodes. In some cases, methods and systems described herein may provide a faster and more efficient way to decode a large number of barcodes to be determined within a pre-set edit distance. For example, a barcode set which comprises at least 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000 or more barcodes. In some cases, the sets of barcodes generated and/or decoded by the methods of the present disclosure may have an edit distance of at least 2, 4, 6, 8, 10 or 12.

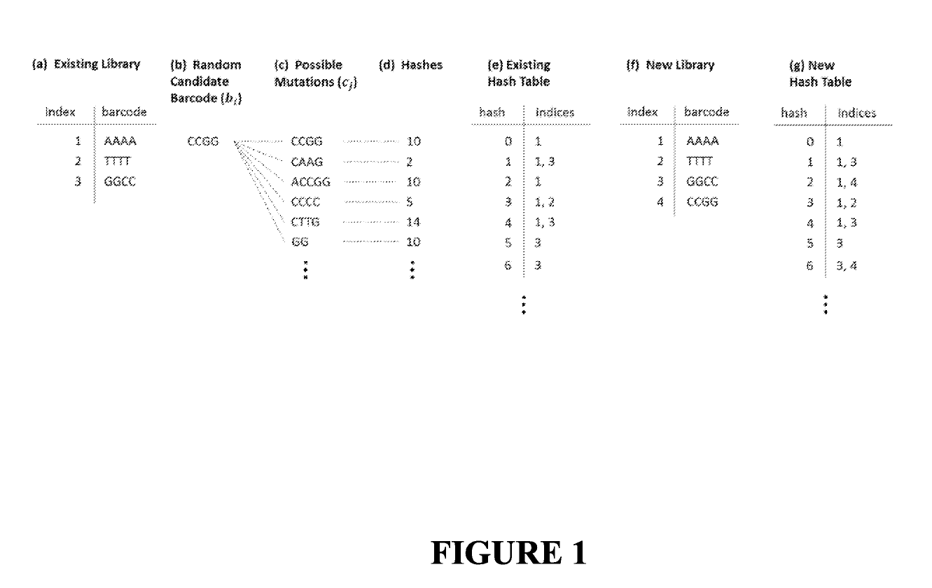

An exemplary procedure for generating a set of barcodes is shown in FIG. 1. First, a set of library barcodes with a pre-set barcode length (n=4) and a library edit distance (d=4) is provided (a). A candidate barcode (bᵢ) is randomly selected from a set of provided candidate barcodes (b) and all possible mutations (cⱼ) of the selected candidate barcode (bᵢ) within a comparison edit distance 2 are calculated and listed (c). A hash function is then utilized to calculate the hash values of each of the mutations cⱼ of the selected candidate barcode bᵢ (d). The hash function used herein is first to convert each of the two rightmost bases in the sequence to a base-4 digit using the dictionary {A:0, C:1, G:2, T:3} and then to convert the resulting 2-digit base-4 number into base-10. For example, for the first mutation listed (i.e., CCGG), the converted base-4 digit of the two rightmost bases is 22, which after the conversion, will result into a base-10 digit 10. In another example, after converting the 2-digit base-4 number of the two rightmost bases (32 for “TG”) in the fifth mutation (i.e., CTTG), the resulting base-10 digit is 14. Subsequently, each of these calculated hash values are compared to the hash values stored in a previously constructed hash table (or creation hash table) (e). If the hash value for one of the mutations cⱼ is already present in the creation hash table and paired with an index (or indices), then the edit distance between the library barcode or library barcodes corresponding to that index (or indices) and the selected candidate barcode bᵢ is calculated, and if this edit distance is less than the library edit distance, the candidate barcode bᵢ is excluded from the set of library barcodes. For example, the edit distance between AAAA and CCGG is calculated because 2 is a hash value for one of its mutations (i.e., CAAG) and is already paired with index 1 in the hash table. After calculation, since the edit distance between AAAA and CCGG is 4, which equals to the pre-set library edit distance, the selected candidate barcode CCGG is not excluded from the set of library barcodes based on this comparison. If the candidate barcode bᵢ is not excluded from the set of library barcodes after iterating through all its mutations cⱼ, then the candidate barcode bᵢ is added to the set of library barcodes and assigned a new library barcode index. Also, a set of mutations c_{g} (not shown in the figure) for the newly added candidate barcode (or the new library barcode) that are within a creation hash table edit distance are generated. In some cases, this creation hash table edit distance can be determined by the formula: creation hash table edit distance=library edit distance−comparison edit distance−1. The creation hash table is then updated accordingly (g) by pairing hash values for each of the mutations c_{g} of the new library barcode with its library barcode index such that the edit distance between each of the mutations c_{g} and the new library barcode is not greater than the creation hash table edit distance.

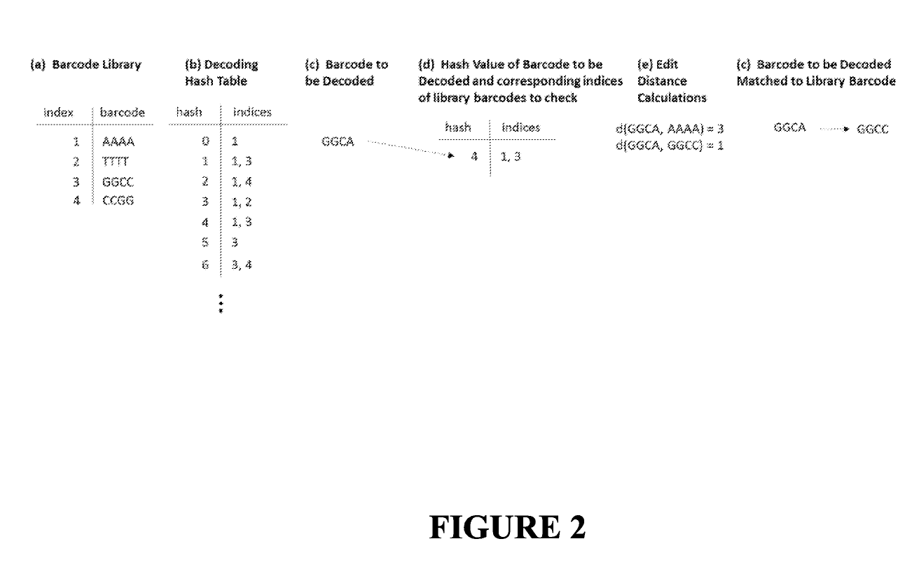

FIG. 2 illustrates an exemplary procedure to decode a set of barcodes to be decoded. First, an indexed set of library barcodes is provided (a). A hash function is used to calculate hash values for each of the library barcodes. As described elsewhere herein, in some cases, the hash function is first to convert each of the two rightmost bases in the sequence to a base-4 digit using the dictionary {A:0, C:1, G:2, T:3} and then to convert the resulting 2-digit base-4 number into base-10. The calculated hash values of library barcodes are then stored and paired to barcode indices associated with each of the library barcodes in a decoding hash table. Then for each library barcode, all its possible mutations within a pre-set resolution edit distance (e.g., 1) are generated. For each of the mutations, its hash value is calculated by using the same hash function as noted above. These calculated hash values are then added to and stored in the decoding hash table, which pairs these hash values with the barcode index of the selected library barcode (b). Once the decoding hash table is generated, a set of barcodes to be decoded is received and a barcode is then selected from the received set (c). For each of the selected barcodes, its hash value is determined and compared to the decoding hash table constructed in step b. If there is a present index (or indices) in the decoding hash table paired with this hash value (d), the edit distance between the corresponding library barcode(s) assigned to that index (or indices) and the selected barcode to be decoded is calculated (e). If the edit distance between the library barcode and the selected barcode is not greater than the above-mentioned resolution edit distance, then the selected barcode is matched to the corresponding library barcode. For example, the edit distances between the selected barcode GGCA and library barcodes AAAA and GGCC are calculated since the hash value of barcode GGCA has already been assigned to library barcode indices 1 and 3 which relate with library barcodes AAAA and GGCC respectively. After calculation, since the edit distance between GGCA and GGCC is 1, which is equal to the pre-set resolution edit distance, the selected barcode GGCA is decoded and matched to the library barcode GGCC. However, if for all of the library barcodes indexed to same hash value, the edit distances between them and the selected barcode to be decoded are greater than the resolution edit distance, then the selected barcode is to be marked as “unresolvable”. For example, if barcode CCAA were received as a barcode to be decoded, its hash value would be firstly calculated. This calculated hash value (i.e., 0) is then compared to the decoding hash table constructed in step b. After comparison, it is determined that this hash value is linked to index 1 in the decoding hash table. Thus, the edit distance between CCAA and the corresponding library barcode AAAA is calculated. Since the edit distance between CCAA and AAAA (i.e., 2) is greater than the resolution edit distance, and there is only one corresponding library barcode for the selected barcode CCAA, the barcode CCAA is marked as “unresolvable”.

As provided in the present disclosure, exemplary methods for generating a set of barcodes may generally include, e.g., listing all possible candidate barcodes in a set of candidate barcodes and initializing a set of library barcodes with a pre-set library edit distance; defining a hash function that may map library barcodes to hash values and initialize a creation hash table which may store these hash values as keys paired to library barcode indices; selecting candidate barcodes once a time from the set of candidate barcodes and for each selected candidate barcode, generating and listing a first set of mutations with a determined comparison edit distance; calculating the hash value for each of the first set of mutation for the selected candidate barcode and if this value has already been assigned an index (or indices) in the creation hash table, calculating the edit distance between the selected candidate barcode and the library barcode(s) assigned to the same index (or indices) in the creation hash table; adding the selected candidate barcode to the set of library barcode if none of the edit distances between the selected candidate barcode and the corresponding library barcode(s) are less than the pre-set library edit distance; generating a second set of mutations of the newly added candidate barcode (or the new library barcode) that are within a creation hash table edit distance, and calculating their hash values; updating the creation hash table by linking the calculated hash values for the second set of mutations to the library barcode index assigned to the new library barcode.

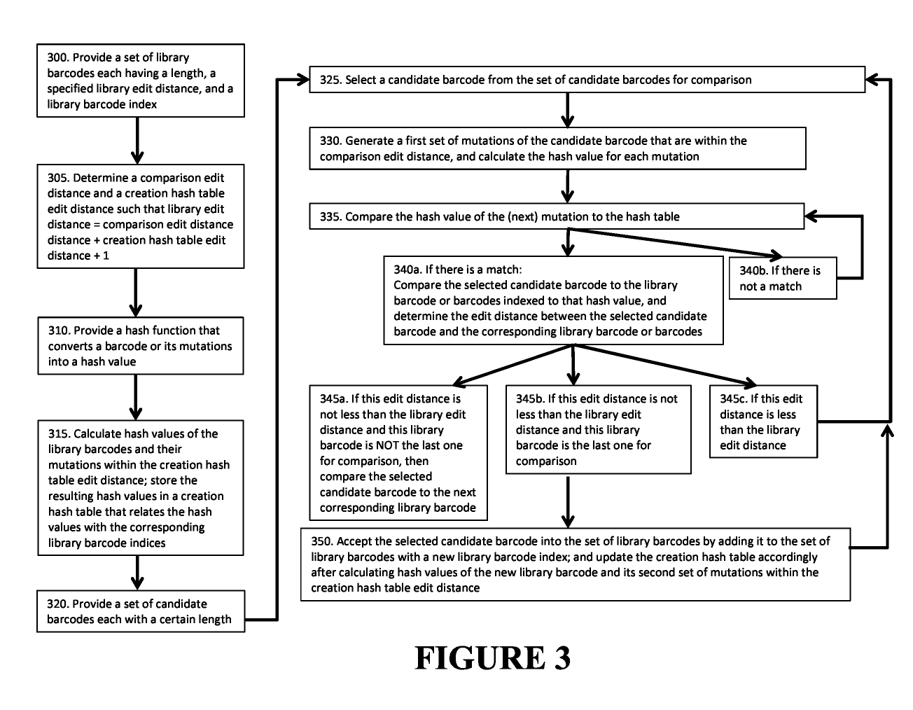

FIG. 3 illustrates an example method for generating a set of barcodes. First, a set of library barcodes may be provided (300). Each barcode included in the set may have a length, a specified library edit distance, and a library barcode index. With the given library edit distance, a comparison edit distance and a creation hash table edit distance may be determined (305). The comparison edit distance can later be used to generate a first set of mutations of the candidate barcodes. The creation hash table edit distance is used here to (i) determine whether a hash value of the barcode can be linked to barcode index or indices in a creation hash table provided later on, and (2) generate a second set of mutations for a candidate barcode if it has been added to the set of library barcodes after comparison. In detail, a hash value of a barcode can be linked to the library barcode index (or indices) in the creation hash table if and only if the edit distance between the barcode and the corresponding library barcode(s) assigned to the library barcode index (or indices) is not greater than the creation hash table edit distance. Notably, for a given library edit distance, the comparison edit distance and the creation hash table edit distance are chosen such that library edit distance=comparison edit distance+creation hash table edit distance+1. In some cases, the comparison edit distance can be determined by using the formula: [library edit distance−1−integer ((library edit distance−1)/2)]. For example, with a given library edit distance 4, the comparison edit distance will be [4−1−1], which is 2. With the given library edit distance (i.e., 4) and the determined comparison edit distance (i.e., 2), the creation hash table edit distance can be easily determined (i.e., 1) by using the formula: creation hash table edit distance=library edit distance−comparison edit distance−1. In some cases, the creation hash table edit distance can be calculated with the formula: integer ((library edit distance−1)/2). For example, with a given library edit distance of 4, the creation hash table edit distance is integer((4−1)/2), which is 1. Once the library edit distance and the creation hash table edit distance are determined, the comparison edit distance is fixed (i.e., 4−1−1=2), based upon the relationship among these three edit distances. According to the determined creation hash table edit distance, mutations of the library barcodes that are within this edit distance may be generated. With a provided hash function (310), hash values of the library barcodes and their mutations are calculated and stored in a creation hash table (315). This creation hash table may then relate the resulting hash values with the corresponding library barcode indices. Following the construction of the creation hash table, a set of candidate barcodes may be provided (320), and each of these candidate barcodes may have a certain length. In some cases, the length of candidate barcodes may be the same as the library barcodes. In some cases, the length of candidate barcodes may be different from the library barcodes. A candidate barcode is then selected from the set of candidate barcodes for comparison (325). A first set of mutations of the selected candidate barcode within the aforementioned comparison edit distance are generated, and for each mutation, its hash value is calculated by the hash function as noted above (330). The calculated hash value for each mutation is then compared (335) to the creation hash table provided in step 315. If there is a match, the selected candidate barcode is then compared to library barcode(s) indexed to the same hash value. Meanwhile, edit distances between the selected candidate barcode and each of the corresponding library barcode(s) are determined (340a). If the determined edit distance is not less than the specified library edit distance, and the corresponding library barcode is not the last one for comparison, then the selected candidate barcode is compared to the next following library barcode until all of the corresponding library barcodes have been compared (345a). For example, if for a selected candidate barcode, it is determined that there are 5 corresponding library barcodes to be compared and after comparison, the edit distance between the selected candidate barcode and first corresponding library barcode is greater than or equal to the library edit distance, then the selected candidate barcode is to be compared to the next following library barcode until either (i) all of the corresponding library barcodes have been compared or (ii) the edit distance between the selected candidate barcode and one of the corresponding library barcodes is less than the library edit distance. If after calculation, it tunes out that all of the edit distances between the selected candidate barcode and the corresponding library barcodes are not less than the pre-set library edit distance (345b), then the selected candidate barcode is added to the set of library barcode as a new library barcode and a new library barcode index is assigned to it in the creation hash table (350). For example, if a selected candidate barcode have 5 mutations in total, and hash values for 2 of its mutations match the existing library indices in the creation hash table, then the selected candidate barcode is compared to all of the corresponding library barcodes that are indexed to the same hash values as those for its two matching mutations. Also, edit distances between the selected candidate barcode and each of the corresponding library barcodes are calculated and compared with the pre-set library edit distance. If after comparison, none of the edit distances between the selected candidate barcode and the corresponding library barcodes are less than the library edit distance, then the selected candidate barcode is accepted into the set of library barcode as a new library barcode and assigned a new library barcode index. Alternatively or additionally, if after comparison (340a), the edit distance between the selected candidate barcode and at least one of the corresponding library barcode(s) is less than the library edit distance, then the selected candidate barcode is not added to the set of library barcodes (345c).

In some cases, one or more screening steps may be included in the methods. Such screening steps may occur in between any of the two steps described above and elsewhere herein. For example, prior to making a comparison between candidate barcodes and library barcodes, at least one of the candidate barcodes may be checked against one or more pre-defined constraints. Non-limiting examples of the constraints may include barcode length, edit distance, homopolymer run limit, GC content of a barcode, melting temperature, forbidden DNA sequences, or combinations thereof. A barcode may be filtered-out or rejected if it fails to meet the pre-defined constraint(s).

Also included in the present disclosure are methods for decoding a set of barcodes to be decoded. An exemplary method for decoding a set of barcodes to be decoded may generally include the steps of: e.g., providing a set of library barcodes and defining a hash function that can convert a barcode and/or its mutations to a hash value; initializing a decoding hash table that stores the converted hash values as keys paired to library barcode indices for the set of library barcodes; selecting a library barcode from the set and for each selected barcode, listing all its possible mutations within a pre-determined edit distance (or resolution edit distance); calculating the hash value for each mutation and adding that value (paired with the library barcode index of the selected library barcode) to the decoding hash table; after iterating through the set of library barcodes, iterating through a set of received barcodes that are to be decoded as follows: (1) calculating the hash value for each of the barcodes to be decoded in the received set; (2) looking up the calculated hash value in the decoding hash table and for each and every index paired to it, comparing the corresponding library barcode(s) to the selected barcode to be decoded and calculating the edit distances between them; and (3) determining whether to match the selected barcode to be decoded to one of the corresponding library barcode or mark it as “unresolvable”, based upon the calculated edit distances obtained in the previous step. For example, if the edit distance between the selected barcode to be decoded and a corresponding library barcode is equal to or less than the resolution edit distance, then the selected barcode to be decoded is matched to that library barcode; or if the edit distances between the selected barcode to be decoded and all its corresponding library barcodes are greater than the resolution edit distance, then the selected barcode to be decoded is marked as “unresolvable”. An updated set of barcodes to be decoded is ultimately constructed after searching through the whole set of received barcodes.

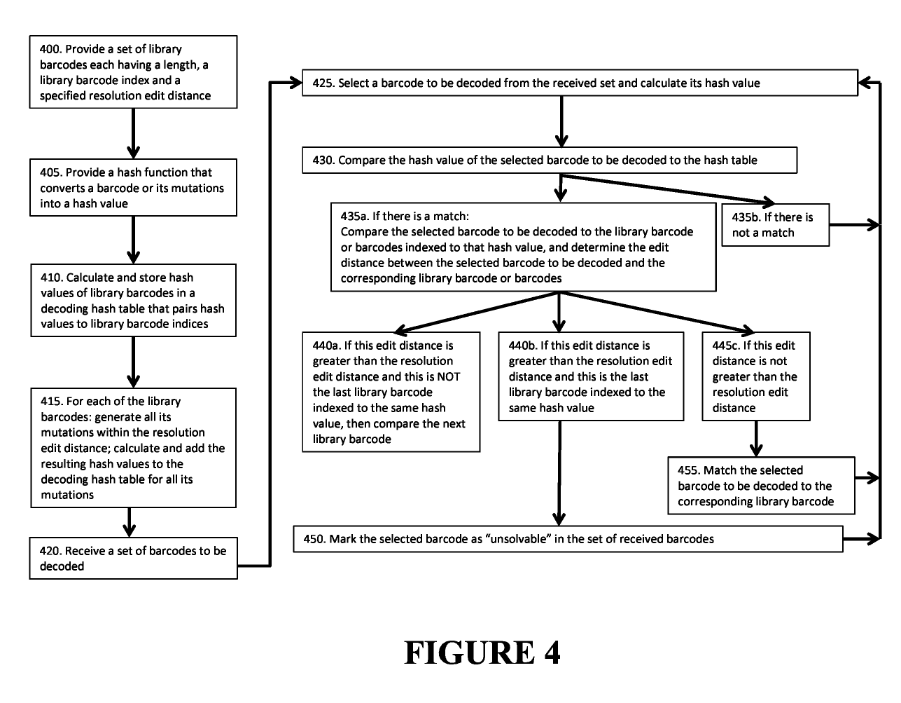

FIG. 4 depicts an exemplary method for decoding a set of candidate barcodes. First, a set of library barcodes is provided (400) wherein each of the library barcodes may have a pre-set length, a specified resolution edit distance and a library barcode index. A hash function that can convert a barcode and/or its mutations into a hash value is then provided (405). With the hash function, the hash value for each of the library barcodes included in the set is calculated and stored in a decoding hash table, which then pairs the hash value of each library barcode to its barcode index (410). After the construction of the decoding hash table, each of the library barcodes listed is then selected and screened as follows: generating all possible mutations of the selected library barcode that are within the resolution edit distance; calculating the hash value for each of its mutations and adding the resulting hash value paired with the library barcode index of the selected library barcode to the decoding hash table (415). Following the completion of searching through the whole set of library barcodes (415), a set of barcodes is received for decoding (or determination) (420). One of the received barcodes is then selected from the set and its hash value is calculated by the same hash function provided in step 405 (425). The calculated hash value is then compared to the decoding hash table to check whether there is match between this hash value and an existing hash value in the decoding hash table (430). If there is not a match, then the selected barcode to be decoded will be returned and the next barcode is selected from the received set and compared (435b). Or, if there is a match, then the selected barcode to be decoded is compared to the corresponding library barcode(s) that is indexed to the same hash value, and an edit distance between the selected barcode and corresponding library barcode(s) is calculated (435a). In cases where more than one corresponding library barcodes are indexed to the same hash value as that of the selected barcode to be decoded, if the determined edit distance between the selected barcode and a corresponding library barcode is greater than the resolution edit distance, while this is not the last corresponding library barcode to be compared, the next following corresponding library barcode will be selected and compared (440a). However, if for all of the corresponding library barcodes, the edit distances between them and the selected barcode to be decoded are greater than the resolution edit distance (440b), the selected barcode to be decoded will be marked as “unresolvable” and the received set of barcodes is updated to include this information. Alternatively, if the edit distance between the selected candidate barcode and a corresponding library barcode is equal to or less than the resolution edit distance (445c), then the selected barcode to be decoded is matched to this corresponding library barcode and the received set of barcodes is updated to reflect the change. In some cases, steps 425-455 may be iterated until (i) all the received barcoded have been compared and decoded, or (ii) a pre-determined number of barcodes have been decoded.

### Characteristics of Barcodes and Set of Barcodes

As provided herein the present disclosure, a barcode (and/or its mutations) can be any sequence of representations that may be used to relate to, associate with or identify a target object. Non-limiting examples of representations may include lines, spacing, colors, images, data, letters, symbols, numbers, characters, numerals, codes, structures, nucleotides, geometric patterns or combinations thereof. In some cases, barcodes may be linear or one-dimensional, for example, barcodes may be represented and recognized by varying the widths and spacing of parallel lines. In some cases, barcodes may be 2-dimensional, for example, they may be made up of rectangles, dots, hexagons and other geometric patterns in two dimensions. In some cases, barcodes may be 3-dimensional, for example, LED-based codes.

The barcodes (and/or their mutations) or sets of barcodes may take any form, tangible or intangible. For example, in some cases, a set of barcodes may comprise a number of computer-generated codes which may be stored in a file. In some cases, a set of barcodes may comprise a plurality of barcodes made of nucleotide or nucleic acid, such as DNA. In cases where barcode are tangible, the set of barcodes may be contained in a reaction mixture. In some cases, the set of barcodes may be stored in a container. A container may be of varied size, shape, weight, and configuration. For example, a container may be round or oval tubular shaped. In some examples, a container may be rectangular, square, diamond, circular, elliptical, or triangular shaped. A container may be regularly shaped or irregularly shaped. Non-limiting examples of types of a container may include a tube, a plate, a chamber, a flow cell, a well, a capillary tube, a cartridge, a cuvette, a centrifuge tube, a chip, or a pipette tip. A container may be constructed of any suitable material with non-limiting examples of such materials that include glasses, metals, plastics, and combinations thereof.

As provided herein, the set of library barcodes may or may not be empty. In cases where the set of library barcodes is not empty, the number of library barcodes contained in the set may vary. In some cases, a large of number of barcodes may be included. In some cases, a small number of barcodes may be included. In some cases, the number of library barcodes in the set of library barcodes can be equal to or less than 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000 barcodes may be included. In some cases, the number of library barcodes in the set of library barcodes can be more than 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000 barcodes. In some cases, the number of the number of library barcodes included in the set of library barcodes may be between any of the two values described herein. For example, 7,500,000 barcodes may be included in the set of library barcodes.

Similarly, the number of barcodes contained in the set of candidate barcodes may be differing. In some cases, a large number of barcodes may be included. In some cases, a small number of barcodes may be included. In some cases, equal to or less than 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000 barcodes may be included. In some cases, more than 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000 barcodes may be included. In some cases, the number of barcodes included in the set of candidate barcodes may be falling into a range of any of the two values described herein. For example, 1,500,000 or 5,500,000 barcodes may be included in the set of candidate barcodes.

The number of barcodes to be decoded contained in a set may vary. In some cases, a large number of barcodes may be included. In some cases, a small number of barcodes may be included. In some cases, equal to or less than 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000 barcodes may be included. In some cases, more than 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000 barcodes may be included. In some cases, the number of barcodes included in the set of barcodes to be decoded may be falling into a range of any of the two values described herein. For example, 1,500,000 or 5,500,000 barcodes may be included in the set.

The length of barcodes (e.g., library barcodes and/or mutations, candidate barcodes and/or mutations, barcodes to be decoded and/or mutations) may vary. In some cases, a barcode may consist of a large number of representations (e.g., letters, symbols, numbers etc.). In some cases, a barcode may consist of a small number of representations. In some cases, a barcode may have a length of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1,000 representations. In some cases, the number of representations contained in a barcode may be less than 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1,000. In some cases, the number of representations contained in a barcode may be more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1,000. In some cases, the number of representations contained in a barcode may be between any of the two values described herein. For example, a barcode may have a length of 22 or 32.

Types of representations contained in a barcode (and/or its mutations) may vary. In some cases, a barcode may consist of a single type of representation, for example, upper-case (or capital) letters or lower-case letters. In some cases, more than one type of representations may be included in a barcode. For example, in some cases, a barcode may comprise both letters and numbers. In some example, a barcode may comprise letters and symbols. In some other examples, a barcode may comprise letters, numbers and symbols.

Length of barcodes contained in the same set of barcodes (e.g., a set of library barcodes, a set of candidate barcodes, a set of barcodes to be decoded etc.) may or may not be the same. In some cases, a set of barcodes may comprise barcodes of the same length. For example, each barcode contained in the same set may have a length of 2, 3, 4 or 5. In some cases, each individual barcode contained in the same set may have their unique length. For example, a set of barcodes may consist of 10 barcodes with lengths of 1, 2, 3, 4, 5, 6, 7, 8, 9 and 10. In some cases, a certain percentage of barcodes contained in the same set may be of the same length. For example, in some cases, equal to or less than 1%, 5%, 10%, 20%, 30%, 40%, 50%, 55%, 60%, 65%, 70%, 75%, 80%, 85%, 90%, 95%, 96%, 97%, 98%, 99%, or 100% of the barcodes in the same set may have the same length. For example, equal to or less than 50%, 90%, or 100% of the barcodes in the same set may have the same length of 4. In some cases, more than 1%, 5%, 10%, 20%, 30%, 40%, 50%, 55%, 60%, 65%, 70%, 75%, 80%, 85%, 90%, 95%, 96%, 97%, 98%, or 99% of the barcodes in the same set may have the same length. For example, more than 50%, 75% or 90% of the barcodes contained in the same set may have a length of 3. In some cases, the percentage of barcodes that have the same length contained in the same set may fall into a range of any of the two values described herein. For example, 99.5% or 99.9% of the barcodes in the same set may be of the same length.

Barcodes contained in different sets may or may not have the same length. For example, in some cases, each of the library barcodes and the candidate barcodes may have the same length. In some cases, each of the library barcodes and the barcodes to be decoded may have the same length. In some cases, barcodes in different sets may have different lengths.

The edit distance between barcodes (e.g., library edit distance, comparison edit distance, creation hash table edit distance, resolution edit distance etc.) may vary. In some cases, a large edit distance may be used, for example, 100. In some cases, a small edit distance may be used, for example, 2 or 4. In some cases, the edit distance may be equal to or less than 1, 2, 3, 4, 5, 6, 7, 8, 9, 10, 15, 20, 25, 30, 35, 40, 45, 50, 60, 70, 80, 90, or 100. In some cases, the edit distance may be at least 1, 2, 3, 4, 5, 6, 7, 8, 9, 10, 15, 20, 25, 30, 35, 40, 45, 50, 60, 70, 80, 90, or 100. In some cases, the edit distance may be between any of the two values described herein, for example, about 12.

As discussed elsewhere in the present disclosure, with a given library edit distance and the formula: library edit distance=comparison edit distance+creation hash table edit distance+1, as long as one of the comparison edit distance and creation hash table edit distance has been determined, the other one is fixed. The order of determining the comparison edit distance and the creation hash table edit distance is highly dependent on the system used to execute the methods and the requirements of applications. For example, as the creation hash table edit distance increases, the memory required to store the creation hash table may increase, therefore, it may be preferred to have a small creation hash table edit distance to allow the entire creation hash table to be stored. Similarly, the time required to update the creation hash table may increase as the creation hash table edit distance increases and the time required to check if a candidate barcode can be accepted into the set of library barcodes may increase as the comparison edit distance increases. Therefore, in some examples, it may be desirable to have a creation hash table edit distance that is greater than or equal to the comparison edit distance, if the number of rejected barcodes is expected to be much greater than the number of accepted barcodes. In some cases, with a given library edit distance, a comparison edit distance is firstly determined, followed by the determination of the creation hash table edit distance. In some cases, the creation hash table edit distance may be determined before the comparison edit distance, with a given library edit distance. In some cases, the comparison edit distance may be 0. In some cases, the creation hash table edit distance may be 0. Also described in the present disclosure is that sets of barcodes may be provided such that each barcode included in may have one or more pre-set or pre-determined characteristics, such as length, type of representations in the barcode, edit distance, and index. In some cases, barcodes contained in the same set may share one or more characteristics, for example, they may have the same length, and/or type of representation, and/or edit distance, and/or index. In some cases, barcodes in the different sets may share one or more characteristics, for example, candidate barcodes may have the same length, and/or type of representation, and/or edit distance, and/or index as library barcodes. In some cases, a certain percentage of barcodes contained in the same set may have one or more identical characteristics, for example, about 5%, 10%, 20%, 30%, 40%, 50%, 60%, 70%, 80%, 90%, or 100% of the library barcodes may share some of the pre-set characteristics. In some cases, each individual barcode may have its unique characteristics.

In cases where a large edit distance d (e.g., library edit distance, comparison edit distance, creation hash table edit distance, resolution edit distance etc.) is employed, in order to decrease the computational time required to generate all possible barcodes, it may be useful to divide the method into several sub-sections, each of which having a smaller edit distance with the sum of all smaller edit distances equal to d. For example, with a given edit distance d (d≧1), a first sub-section of the method may comprise storing the barcodes and all possible mutations with edit distance 1 from the barcode in the hash table. Then for each new barcode (generated mutations), a second sub-section of the method may include the step of generating all possible barcodes whose edit distance from the new barcode is less than (d−1).

In cases where barcodes are received for decoding, a determination error rate may be used and the decoded set of barcodes may be required to be below a pre-determined threshold of the determination error rate. As described elsewhere herein, by “determination error rate” we mean the percentage of received barcodes to be decoded which are incorrectly decoded. For example, if a total of 1,000 barcodes are decoded and 2 of them are incorrectly decoded, then the determination error rate is 0.2%. Depending upon the method design and the application, the determination error rate may vary. In some cases, the determination error rate may be equal to or less than 10%, 5%, 2.5%. 1%, 0.5%, 0.25%, 0.1%, 0.05%, 0.025%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, 0.0001%, 0.00005%, 0.000025%, 0.00001%, 0.000005%, 0.0000025%, or 0.000001%. In some cases, the determination error rate may be between any of the two values described herein. For example, the determination rate may be about 0.0015% or 0.00095%.

Similarly, once a set of barcodes are generated, prior to its application (e.g., DNA sequencing), an “error rate” may be determined against the set of barcodes and only the set of barcodes having the error rate that is below a pre-determined threshold (e.g., 0.1%, 0.01%, or 0.001%) may be released for further use. As used herein, the “error rate” refers to the rate at which a generated barcode is incorrectly identified as a different barcode. For example, if a generated set of barcodes comprises a total of 10,000 barcodes and 5 of which are incorrectly identified as different barcodes, then the error rate of such set of barcodes is 0.05%. Depending upon the applications of the generated barcodes, the error rate may vary. In some cases, the error rate of the generated set of barcodes may be equal to or less than 10%, 5%, 2.5%. 1%, 0.5%, 0.25%, 0.1%, 0.05%, 0.025%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, 0.0001%, 0.00005%, 0.000025%, 0.00001%, 0.000005%, 0.0000025%, or 0.000001%. In some cases, the error rate may be between any of the two values described herein, for example, about 0.0015% or 0.00095%. In cases only a specific type of edits (i.e., substitutions, insertions or deletions) if of interest, the error rate may further refer to a substitution error rate, an insertion error rate, or a deletion error rate, and the set of generated barcodes may be tested against one or more of the error rates prior to any further application.

As will be appreciated, the characteristics of barcodes and sets of barcodes may be altered or adjusted, based upon the requirements of applications, for example, size of barcodes sets, determination error rate, total execution time, available memory space etc. For example, in some cases, it may be desirable to generate a set of barcodes comprising at least 1,000,000 barcodes in less than 20 hours. To meet this requirement, it may be needed to adjust at least one of the characteristics of the systems including but not limited to library barcode length, candidate barcode length, length of barcodes to be decode, library edit distance, comparison edit distance, creation hash table edit distance, resolution edit distance, type of hash function, size of initial set of library barcodes (if applicable), size of initial set of candidate barcodes (if applicable), barcode search strategy (i.e., randomly, semi-randomly, in order etc.).

Also provided in the present disclosure is that barcodes can be listed or searched randomly or in an order. For example, in some cases, barcodes may be listed in order, such as in lexicographical order, in alphabetical order, in chronological order, or in dictionary order. In some cases, the listed barcodes can be search through lexicographically, alphabetically, or chronologically. In some cases where a method comprises a list or a set of lexicographically ordered barcodes, the method may be referred to as Algorithm with Hash Table (or AHT). In some cases, depending upon the applications, listing or selection of the barcodes may be in a random order, for example, if an expected execution time or time complexity of the method (or algorithm) is required in an application. In some cases, in order to reduce the execution time, it may be desirable to reduce the number of barcodes to be searched through and compared. Therefore, instead of searching through all barcodes in an ordered manner (e.g., lexicographically), the barcodes may be searched through in a random order. In some cases, some pre-set criteria may be used to gauge and control the progress of the searching. For example, the search of the barcodes may be ceased until either (1) all of the barcodes in the set has been searched through, or (2) a pre-determined set size has been reached. In cases where the barcodes are searched randomly in a method, the method may be referred to as Randomized Algorithm with Hash Table (or RAHT).

### Computer-Implemented Systems and Methods

Also provided in the present disclosure are systems and computer-implemented methods for barcode creating and decoding as disclosed elsewhere herein. Generally, the computer-implemented systems or methods may be configured to be capable of receiving a request from a user, executing program modules to implementing a method, performing a task, and outputting the results to a recipient. In some cases, examples of requests or received information may include but not limited to: size of the set of library barcodes (or the number of library barcodes included in the set), size of the set of candidate barcodes (or the number of the candidate barcodes included in the set), size of the set of barcodes to be generated (or the number of barcodes included in the generated set of barcodes), length(s) of the library barcodes; length(s) of the mutations of the library barcodes, length(s) of the candidate barcodes, length(s) of the mutations of the candidate barcodes, library edit distance, comparison edit distance, creation hash table edit distance, type of hash function(s) to be used, barcode search strategy, type of representations included in each of the barcodes and its mutations, number of representations of representations included in each of the barcodes and its mutations, execution time, unit execution time, biological constraints, chemical constraints, or combinations thereof. Exemplary outputted results may comprise a set of generated barcodes and information regarding the set and each of the barcodes included in the set such as the number of barcodes generated, barcode length(s), type of representations in each of the generated barcodes, library edit distance, comparison edit distance, creation hash table edit distance, type of hash function used to determine the hash values of the barcodes and their mutations, criteria used to screen and generate the barcodes etc.

In some cases, examples of requests or received information may include but not limited to: size of the set of library barcodes (or the number of library barcodes included in the set), size of the set of barcodes to be decoded (or the number of barcodes that are to be decoded), size of length(s) of the library barcodes, length(s) of the barcodes to be decoded, length(s) of the mutations of the library barcodes, resolution edit distance, type of hash function(s) to be used, barcode search strategy, type of representations included in each of the barcodes and its mutations, number of representations included in each of the barcodes and its mutations, execution time, unit execution time, biological constraints, chemical constraints, or combinations thereof. Example outputted results may comprise the set of barcodes that has been examined and decoded, along with the information with respect to the set of decoded barcodes and each of the barcodes included in the set, e.g., the number of barcodes included in the set, length(s) of the barcodes, type of representation included in each of the barcodes, type of hash function utilized to determine the hash values of the barcodes, barcode search strategy, resolution edit distance, and criteria used to examine and decode barcodes etc.

For example, in some embodiments, the present disclosure may provide a system for using a set of barcodes with a pre-set edit distance, which comprises: (i) a computer configured to receive a request to generate a set of barcodes with a pre-determined edit distance; (ii) one or more processors capable of implementing a method for generating a set of barcodes upon execution of program codes; and (iii) a report generator that may send the information regarding the results to a recipient. In some other embodiments, a system for using a set of decoded barcoded may be provided. The system may comprise: (i) a computer configured to receive a request to decode a set of received barcodes; (ii) one or more processors capable of implementing a method for decoding a set of barcodes upon execution of stored program codes; and (iii) a report generator that may send the information regarding the results to a recipient.

Various types of hash functions such as cyclic redundancy checks, checksum functions, Non-cryptographic hash functions and cryptographic hash functions may be utilized as provided in the present disclosure. Non-limiting examples of hash function may include BSD checksum, checksum, crc16, crc32, crc32 mpeg2, crc 64, SYSV checksum, sum (Unix), sum8, sum16, sum24, sum32, fletcher-4, fletcher-8, fletcher-16, fletcher-32, Adler-32, xor8, Luhn algorithm, Verhoeff algorithm, Damm algorithm, Pearson hashing, Buzhash, Fowler-Noll-Vo hash function (FNV Hash), Zobrist hashing, Jenkins hash function, Java hashCode, Bernstein hash, elf64, MurmurHash, SpookyHash, Jenkins hash function, CityHash 64, xxHash, BLAKE-256, BLAKE-512, ECOH, FSB, GOST, Grøst1, HAS-160, HAVAL, JH, MD2, MD4, MD5, MD6, RadioGatún, RIPEMD-64, RIPEMD-160, RIPEMD-320, SHA-1, SHA-224, SHA-256, SHA-384, SHA-512, SHA-3, Skein, SipHash, Snefru, Spectral Hash, SWIFFT 512 bits hash, Tiger, Whirlpool, or combinations thereof. For example, as provided elsewhere herein, a hash function may first convert two rightmost representations in a barcode to a base-4 number and subsequently convert the resulting base-4 number into a base-10 number. In some examples, a greater number of representations (e.g., 10 or 14 rightmost representations of the barcode) may be initially converted to a base-4 digit by the hash function and then transformed into a base-10 digit. Any module capable of accepting a user request may be used. The module may comprise, for example, a device that comprises one or more processors. Non-limiting examples of devices may include a desktop computer, a laptop computer, a tablet computer, a cell phone, a smart phone, a personal digital assistant (PDA), a video-game console, a television, a music playback device, a video playback device, a pager, and a calculator. Processors may be associated with one or more controllers, calculation units, and/or other units of a computer system, or implanted in firmware as desired. If implemented in software, the routines (or programs) may be stored in any computer readable memory such as in RAM, ROM, flash memory, a magnetic disk, a laser disk, or other storage medium. Likewise, this software may be delivered to a device via any delivery method including, for example, over a communication channel such as a telephone line, the internet, a local intranet, a wireless connection, etc., or via a transportable medium, such as a computer readable disk, flash drive, etc. The various steps may be implemented as various blocks, operations, tools, modules or techniques which, in turn, may be implemented in hardware, firmware, software, or any combination thereof. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

The module may be configured to receive the user request directly (e.g. by way of an input device such as a keyboard, mouse, or touch screen operated by the user) or indirectly (e.g. through a wired or wireless connection, including over the internet). In some embodiments, a module may include a user interface (UI), such as a graphical user interface (GUI), that is configured to enable a user provide a request. In some cases, a GUI may include textual, graphical and/or audio components. In some cases, a GUI may be provided on an electronic display, including the display of a device comprising a computer processor. Such a display may include a resistive or capacitive touch screen.

Non-limiting examples of users may include a client, a customer, medical personnel, a clinician (e.g., a doctor, a nurse, and a laboratory technician etc.), laboratory personnel (e.g., a hospital laboratory technician, a research scientist, a pharmaceutical scientist), a clinical monitor for a clinical trial, or others in the health care industry, a company, a local or offsite facility, an electronic system (e.g., one or more computers and/or one or more computer servers storing etc.), and a computer-readable medium.

The information may be outputted to various types of recipients. The recipients may or may not be the same as the users. Non-limiting examples of such recipients may include a user who sends the request, a client, a customer, a physician, a clinical monitor for a clinical trial, a nurse, a researcher, a laboratory technician, a representative of a pharmaceutical company, a health care company, a biotechnology company, a hospital, a human aid organization, a health care manager, a public health worker, other medical personnel, other medical facilities, an electronic system (e.g., one or more computers and/or one or more computer servers storing) and a computer-readable medium.

Common forms of computer-readable media may include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more barcode sequences of one or more instructions to a processor for execution.

Information may be outputted via any suitable means. In some embodiments, such information may be provided verbally to a recipient. In some embodiments, such information may be provided in a report. A report may include any number of desired elements, with non-limiting examples that include information regarding the objectives, lists or sets of original data (e.g., set of original library barcodes, set of original candidate barcodes, set of potentially changed barcodes etc.), lists or sets of processed data (e.g., updated set of library barcodes, updated set of candidate barcodes, update list of potentially changed barcodes etc.), detailed information of the data (e.g., barcode length, edit distance, type of representations in barcodes etc.), detailed information of method (e.g., hash function), and the like, and combinations thereof. The report may be provided as a printed report (e.g., a hard copy) or may be provided as an electronic report. In some embodiments, including cases where an electronic report is provided, such information may be outputted via an electronic display, such as a monitor or television, a screen operatively linked with a unit used to obtain the amplified product, a tablet computer screen, a mobile device screen, and the like. Both printed and electronic reports may be stored in storage devices such that they are accessible for comparison with future reports. Non-limiting examples of storage devices may include: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, or any other memory chip or cartridge.

Moreover, a report may be transmitted to the recipient at a local or remote location using any suitable communication medium including, for example, a network connection, a wireless connection or an internet connection. In some embodiments, a report can be sent to a recipient's device, such as a personal computer, phone, tablet, or other device. The report may be viewed online, saved on the recipient's device, or printed. A report can also be transmitted by any other suitable means for transmitting information, with non-limiting examples that include mailing a hard-copy report for reception and/or for review by a recipient. In some cases, the report may be retrieved from a third-party data source.

### Execution Time of Methods

As described elsewhere herein, the present disclosure provides faster and more efficient methods for generating and decoding a large number of barcodes with high accuracy, e.g., generating and/or decoding a set of 50 million barcodes with an accuracy of at least about 50%, 55%, 60%, 65%, 70%, 75%, 80%, 82%, 84%, 86%, 88%, 90%, 91%, 92%, 93%, 94%, 95%, 96%, 97%, 98%, 99%, 99.5%, 99.9%, 99.99%, or 99.999%. In some cases, generating and/or decoding accuracy may be dependent upon a number of factors, e.g., edit distance, barcode length, number of barcodes to be generated or decoded, per-base substitution rate, and/or user-defined constraints.

For example, methods provided herein may be sued to generate a set of 1,000,000 or more barcodes in less than 24 hours. In another example, methods of the present disclosure may be used for decoding a set of 1,000,000 or more barcodes within 5 minutes. In general, the execution time for a method to generate or decode a set of barcodes may vary, depending upon, requirements of applications, for example, characteristics of barcodes and barcode set that are to be generated or decoded. Non-limiting examples of characteristics of barcodes and barcode set may include length of barcode, edit distance (e.g., library edit distance, comparison edit distance, resolution edit distance etc.) between barcodes, size of barcode set (i.e., number of barcodes included in a set), maximum determination error rate, pre-defined constraints or combinations thereof.

In some cases, it may be desirable to generate or decode a set of barcodes within a certain amount of time. For example, the execution time for a method to generate or decode a set of barcodes may be less than 500 hours, 250 hours, 100 hours, 80 hours, 60 hours, 50 hours, 40 hours, 30 hours, 25 hours, 20 hours, 15 hours, 10 hours, 9 hours, 8 hours, 7 hours, 6 hours, 5 hours, 4 hours, 3 hours, 2 hours, 1 hour, 3,000 s, 2,000 s, 1,000 s, 900 s, 800 s, 700 s, 600 s, 500 s, 400 s, 300 s, 200 s, 100 s, 75 s, 50 s, 25 s, 10 s, 0.75 s, 0.5 s, 0.25 s, 0.1 s, 0.075 s, 0.05 s, 0.025 s, 0.01 s, 0.0075 s, 0.005 s, 0.0025 s, 0.001 s, 0.00075 s, 0.0005 s, 0.00025 s, 0.0001 s, 0.000075 s, 0.00005 s, 0.000025 s, 0.00001 s, 0.0000075 s, 0.000005 s, 0.0000025 s, 0.000001 s, 0.00000075 s, 0.0000005 s, 0.00000025 s or 0.0000001 s. In some cases, the execution time may be between any of the two values described herein. For example, the execution time may be 5,000 s.

In some cases, methods provided herein may generate or decode a large number of barcodes within a certain unit execution time. By “unit execution time” we mean the average time period used to generate or decode an individual barcode within a set, which can be determined by dividing the execution time by the total number of barcodes generated or decoded. In some cases, the unit execution time may equal to or less than 1,000 s, 750 s, 500 s, 250 s, 100 s, 75 s, 50 s, 25 s, 10 s, 9 s, 8 s, 7 s, 6 s, 5 s, 4 s, 3 s, 2 s, 1 s, 0.9 s, 0.8 s, 0.7 s, 0.6 s, 0.5 s, 0.4 s, 0.3 s, 0.2 s, 0.1 s, 0.09 s, 0.08 s, 0.07 s, 0.06 s, 0.05 s, 0.04 s, 0.03 s, 0.02 s, 0.01 s, 0.009 s, 0.008 s, 0.007 s, 0.006 s, 0.005 s, 0.004 s, 0.003 s, 0.002 s, 0.001 s, 0.0009 s, 0.0008 s, 0.0007 s, 0.0006 s, 0.0005 s, 0.0004 s, 0.0003 s, 0.0002 s, 0.0001 s, 0.00009 s, 0.00008 s, 0.00007 s, 0.00006 s, 0.00005 s, 0.00004 s, 0.00003 s, 0.00002 s, 0.00001 s, 0.000009 s, 0.000008 s, 0.000007 s, 0.000006 s, 0.000005 s, 0.000004 s, 0.000003 s, 0.000002 s, 0.000001 s, 0.0000009 s, 0.0000008 s, 0.0000007 s, 0.0000006 s, 0.0000005 s, 0.0000004 s, 0.0000003 s, 0.0000002 s, or 0.0000001 s. In some cases, the unit execution time may fall into a range of any of the two values described herein. For example, the unit execution time may be 0.012 s or 0.0057 s.

### Kits

Kits of the present disclosure are provided herein. As described elsewhere herein, the barcodes may take any form of existence, for example, made up of nucleotides or nucleic acids. In cases where barcodes are made of nucleotides or nucleic acids, the barcodes may be contained in a reaction mixture. The reaction mixture may be further packaged in a kit. In some cases, the kit may comprise one or more additional reagents, for example, reagents for amplification reactions. Non-limiting examples of reagents may comprise polymerase enzymes, nucleoside triphosphates or their analogues, primer sequences, buffers, and combinations thereof. In some cases, additional information that may be used to facilitate the use of the barcodes may be included in the kit, for example, a source identifier or an information link that may aid in accurately and timely retrieving the source or information of provided barcodes. The kit may also contain instructions for the use of kit such as, for example, methods of generating a set of barcodes, methods of using the a generated set of barcodes, methods of decoding a set of potentially changed barcodes, and methods of using a set of decoded potentially changed barcodes.

### Barcodes and Sequencing

Methods and systems provided in the present disclosure may find useful in a wide variety of contexts, for example, nucleic acid sequencing in biotechnology. Non-limiting examples of sequencing techniques may involve basic methods such as Maxam-Gilbert sequencing and chain-termination (or Sanger sequencing) methods, de novo sequencing methods including shotgun sequencing and bridge PCR, next-generation methods including polony sequencing, 454 pyrosequencing, Illumina sequencing, SOLiD sequencing, Ion Torrent semiconductor sequencing, Heloscope single molecule sequencing and others.

Barcodes created and checked by the methods described in the present disclosure may be used for tagging, tracking, and identifying any sample or species in sequencing. A sample or species can be, for example, any substance used in sample processing, such as a reagent or an analyte. Exemplary samples may include whole cells, chromosomes, polynucleotides, organic molecules, proteins, polypeptides, carbohydrates, saccharides, sugars, lipids, enzymes, restriction enzymes, ligases, polymerases, barcodes, adaptors, small molecules, antibodies, fluorophores, deoxynucleotide triphosphate (dNTPs), dideoxynucleotide triphosphates (ddNTPs), buffers, acidic solutions, basic solutions, temperature-sensitive enzymes, pH-sensitive enzymes, light-sensitive enzymes, metals, metal ions, magnesium chloride, sodium chloride, manganese, aqueous buffer, mild buffer, ionic buffer, inhibitors, oils, salts, ions, detergents, ionic detergents, non-ionic detergents, oligonucleotides, nucleotides, DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, proteases, nucleases, protease inhibitors, nuclease inhibitors, chelating agents, reducing agents, oxidizing agents, probes, chromophores, dyes, organics, emulsifiers, surfactants, stabilizers, polymers, water, pharmaceuticals, radioactive molecules, preservatives, antibiotics, aptamers, and the like.

In the present disclosure, barcode used in sequencing applications may comprise a plurality of barcodes made up of a number of nucleotides. In some cases, the barcodes may be made up of nucleic acids. For example, the barcodes may be made up of DNA, RNA, or DNA-RNA hybrids. In cases where the barcodes are made up of nucleotides or nucleic acid, representations used in barcodes may comprise letters (including upper-case and lower-case letters) or characters which represent one of the four nucleotide subunits of a DNA or a RNA strand (i.e., “A”, “T”, “G”, “C” and “U”). For example, in some cases, barcodes may be denoted by “aaccagttc”, “TGGAATTCG”, or “AACCAGUUC”.

The barcode sequence (e.g., library barcode and/or its mutations, candidate barcode and/or its mutations, and/or barcode to be decoded and/or its mutations) described herein may be of any length, depending on the application. In some cases, a barcode may have a length equal to or less than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 85, 90, 95, 100, 200, 300, 400, 500, 600, 700, 800, 900, or 1,000. For example, a barcode may have a length of 4, 15 or 18. In some cases, a barcode may have a length greater than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 85, 90, 95, 100, 200, 300, 400, 500, 600, 700, 800, 900, or 1,000. For example, a barcode may have a length greater than about 3. In some cases, a barcode may have a length in between any of the two values described herein. For example, a barcode may have a length of 21 or 33.

Barcodes contained in the same set may or may not have the same length. For example, in some cases, each barcode contained in the same set may be of the same length. In some cases, none of the barcode in the same set may have the same length. In some cases, a certain percentage of the barcodes contained in the same set may have the same length. For example, 5%, 10%, 15%, 20%, 30%, 40%, 50%, 60%, 70%, 80%, 90%, or 100% of the barcodes in the same set may have the same length.

Barcodes belonging to different sets may or may not have the same length. For example, in cases where both sets of library barcodes and candidate barcodes are provided, each of library barcodes and candidate barcodes may have the same length. In some examples, each of the library barcodes and candidate barcodes may have a length of 4. In another example, when a set of barcodes is received for decoding, each of the received barcode may have the same length as the library barcodes, for example, a length of 10 or 20.

Number of barcodes contained in a certain set of barcodes (e.g., a set of library barcodes, a set of candidate barcodes, a set of barcodes to be decoded etc.) may vary, depending upon, for example, the type of application, the length of barcodes, the expected execution time of the task etc. In some cases, a large number of barcodes may be used, for example, 10,000,000. In some cases, a small number of barcodes may be used, for example, 100. In some cases, the number of barcodes may be equal to or less than 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000. In some cases, the number of barcodes may be at least 1, 10, 100, 1,000, 10,000, 50,000, 100,000, 250,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000. In some cases, the number of barcodes may fall into a range of any of the two values described herein. For example, about 1,500,000 or 5,500,000 barcodes may be used.

In some cases, some additional information or annotation may be associated with the barcodes. Non-limiting examples of such information or annotations may include adapters, linkers, strand of nucleic acid sequences, complete nucleic acid sequences (e.g., DNA sequences, RNA sequences etc.), source identifiers, information links, or combinations thereof.

When using barcode sequences for certain applications, some biological and chemical constraints may be considered in the barcode design. Examples of possible constraints may include, but not limited to, GC and/or AT content in a particular range, ATG content in a certain range, nucleotide repeats, complexity, edit distance to reverse complement, presence of forbidden sequences (e.g., sequences having a certain number of nucleotides in a row from the group consisting of G and C or A and T, sequences having a start codon), melting temperature, homopolymer runs beyond a certain range (or homopolymer limit), propensity for the formation of intramolecular secondary structures (e.g., hairpin structures), propensity for intermolecular annealing, exclusion of particular motifs (e.g., when using restriction enzymes), low similarity to genomic DNA, low similarity to mRNA sequence, and the like, and the combinations thereof. Barcodes that fail to meet one or more of the constraints may be filtered out or removed before one or more steps of the methods, e.g., prior to performing a comparison of a candidate barcode to creation hash table, or decoding hash table. For example, in some cases, before comparison, candidate barcodes with a cutoff value of G+C content of about 70% are removed. In some examples, it may be designed to remove from the list all barcodes that contain homopolymers with a length of greater than a cutoff value (e.g., 3). In some examples, it may be configured to remove from list all barcodes for which composite forward primers potentially form heteroduplexes with reverse primer of length greater than a cutoff value (e.g., 7 basepairs).

As described elsewhere herein the present disclosure, in some applications, it may be desirable to have a set of barcodes with a determination error rate less than an acceptable value, or a threshold. The systems and methods described herein may be modified and reiterated until the determination error rate falls below the acceptable value. In some cases, the threshold may be equal to or less than 30%, 20%, 15%, 10%, 7.5%, 5%, 2.5%. 1%, 0.5%, 0.25%, 0.1%, 0.05%, 0.025%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, 0.0001%, 0.00005%, 0.000025%, 0.00001%, 0.000005%, 0.0000025%, or 0.000001%. In some cases, the threshold may be between any of the two values described herein. For example, it may be required to have a determination error rate less than about 0.0015% or 0.00095%.

### EXAMPLES

### Example 1: Generating DNA Barcodes

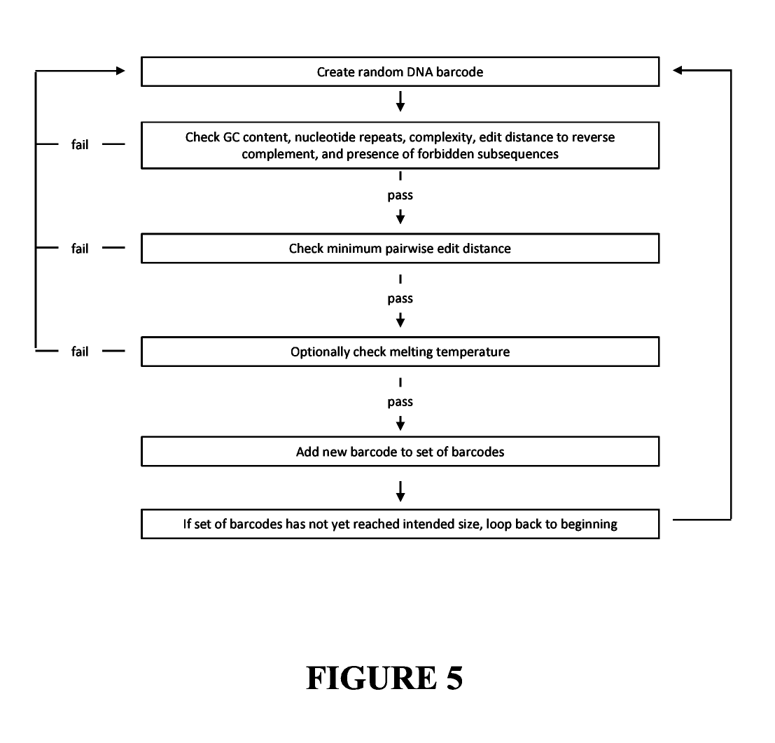

As shown in FIG. 5, for each set of barcodes to be generated, a number of parameters and/or user-defined constraints are entered, e.g., number of barcodes to be generated, a barcode length, a minimum pairwise edit distance, a homopolymer run limit, an acceptable range of barcode GC content, a minimum for the edit distance between a barcode and its reverse complement, and a list of forbidden DNA subsequences. To generate a set of DNA barcodes, a random barcode of the specified length is iteratively created and checked against all of the user-defined constraints except the minimum pairwise edit distance. If the barcode meets all of these user-defined constraints, the barcode is then checked to make sure it meets the minimum pairwise edit distance requirement.

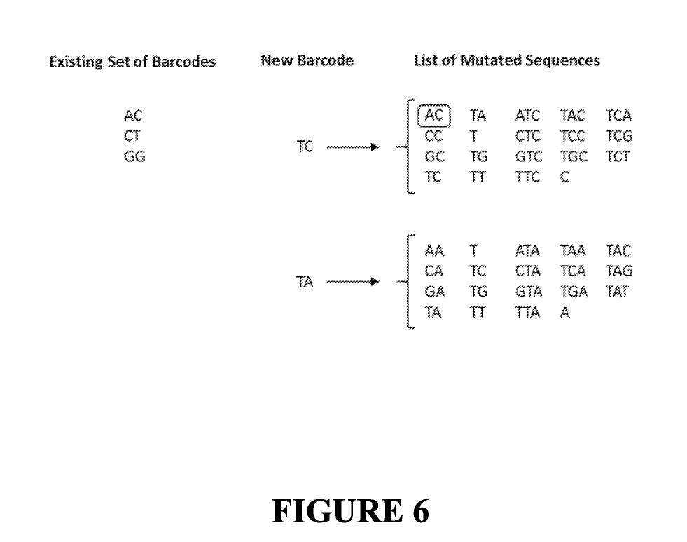

In order to ensure a new barcode does not violate the minimum pairwise edit distance requirement, all possible DNA sequences whose edit distance from the new barcode is less than the minimum pairwise edit distance of the set are listed. If none of these mutated sequences are in the set of DNA barcodes, then the edit distance between the new barcode and every other barcode in the set of DNA barcodes is at least the minimum pairwise edit distance (FIG. 6). Since the set of DNA barcodes are stored as a hash table, so the time required for checking if each mutated sequence is in the set of DNA barcodes is independent of the size of the set.

As shown in FIG. 6, the barcode length is 2, and the minimum pairwise edit distance is also 2. Barcodes AC, CT, and GG are already added to the set. For new barcode TC, all possible DNA sequences within edit distance 1 are listed. Since the edit distance between TC and AC is only 1, which is less than the minimum pairwise edit distance (i.e., 2), TC is not added to the set. For new barcode TA, none of the sequences in the list of its mutated sequences appear in the existing set of barcodes, which indicates that the edit distance between TA and each of the DNA barcodes in the existing set is at least 2, so TA can be added to the set of barcodes.

After a barcode has been checked for minimum pairwise edit distance, the melting temperature of the secondary structure of the barcode can be checked and the barcode may be filtered out if the melting temperature exceeds a user-entered cutoff. Various methods can be used to calculate the melting temperature, e.g., UNAFold software package. In some cases, a sodium concentration and left and right adaptors to be added to the left and right of the barcode are entered for the secondary structure melting temperature calculation.

### Example 2: Methods and Elapsed Time for Generating DNA Barcodes

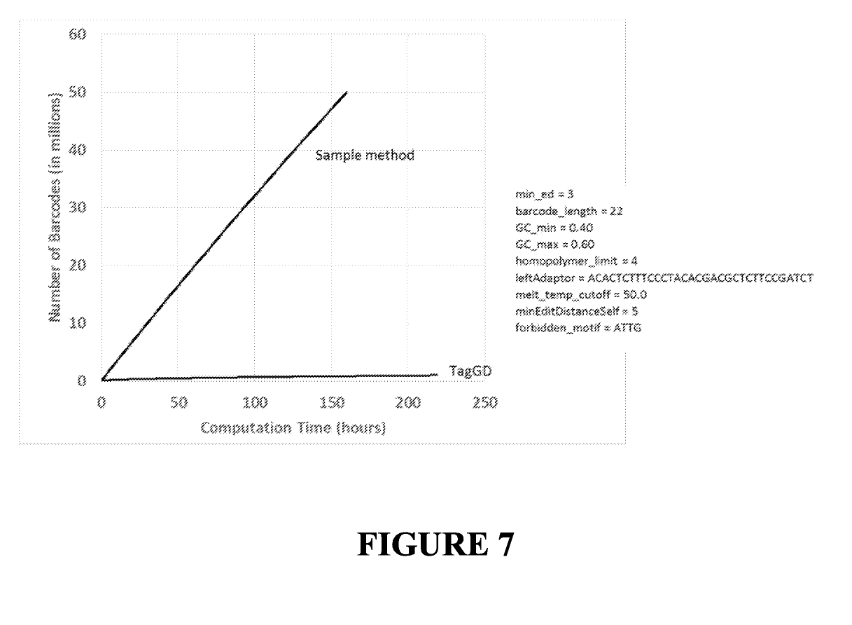

An exemplary method of the present disclosure and a different method (e.g., TagGD) were employed to produce sets of DNA barcodes with a minimum pairwise edit distance of 3 of the same machine (a Linux machine with 12 CPU cores and 24 GB RAM). FIG. 7 plots the times required to build sets of DNA barcodes versus the barcode set sizes for each method. With the method of the present disclosure, a set of 50 million barcodes was produced in about 160 hours; while it took about 219 hours to produce a set of 1 million barcodes with TagGD. Additionally, as TagGD produced the set of 1 million barcodes, the time required for each new barcode increased from about 0.017 seconds per barcode in the beginning to more than 1.5 seconds per barcode by the end. In contrast, as the method of present disclosure generated the set of 50 million barcodes, the time required for each new barcode only increased from about 0.009 seconds per barcode in the beginning to about 0.012 seconds per barcode by the end.

### Example 3: Methods and Elapsed Time for Decoding DNA Barcodes

The exemplary method as described above in Example 2 and its generated set of 50 million DNA barcodes were utilized to decode 100 million simulated DNA sequencing reads with various per-base substitution rates (Table 1). The set of 50 million DNA barcodes with minimum pairwise edit distance 3 was firstly used to simulate 100 million reads with per-base substitution rates of 0.2%, 1%, and 5%. The exemplary method as described above was then employed to decode the reads, with up to 1 error correction. Once the decoding process was completed, the number of reads which were decoded correctly, the number of reads which were decoded incorrectly, and the number of reads which could not be decoded because they were not within edit distance 1 of a barcode in the set of barcodes were counted. With the method of the present disclosure, the decoding process took less than 2 hours to process 100 million DNA reads when correcting up to 1 error per barcode. In comparison, TagGD required more than 1.5 hours to decode just 10,000 reads given a set of just 10 million DNA barcodes.

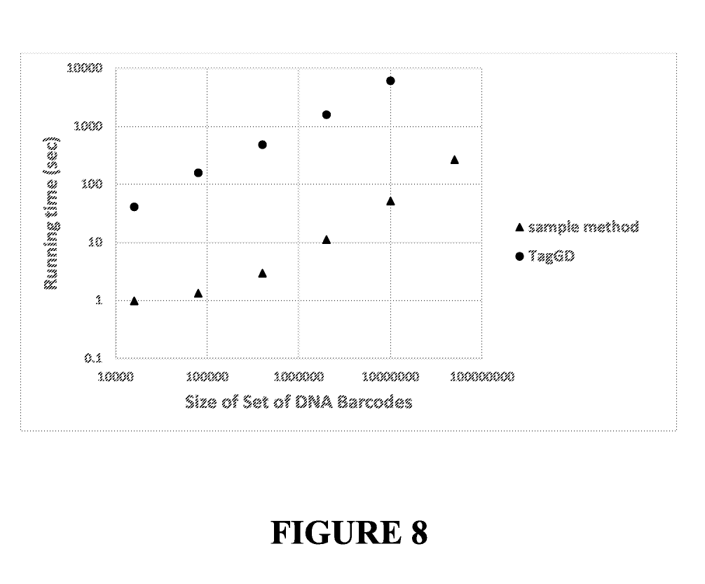

In order to compare the decoding programs from the two methods (i.e, exemplary method of the present disclosure and TagGD), a total of 10,000 simulated DNA reads was decoded by using each of the methods given a range of DNA barcode set sizes (i.e., 16,000 to 50 million) and the results are plotted in FIG. 8. As shown in FIG. 8, method of the present disclosure is well equipped to decode reads within the whole range of DNA barcode set sizes, while TagGD is unable to finish decoding millions of reads given a set of tens of millions of DNA barcodes.

TABLE 1
Decoding accuracy vs. per-base substitution rate

| | Number of | Number | Number of | |
|---|---|---|---|---|
| Per-base | reads | of reads | reads which | Running |
| substitution | decoded | decoded | could not | time |
| rate | correctly | incorrectly | be decoded | (sec) |
| 0.2% | 99,910,185 | 25 | 89,790 | 1,955 |
| 1% | 97,976,584 | 532 | 2,022,884 | 2,938 |
| 5% | 69,825,096 | 7,458 | 30,167,446 | 6,106 |

### Example 4: Methods and Size of Barcode Set

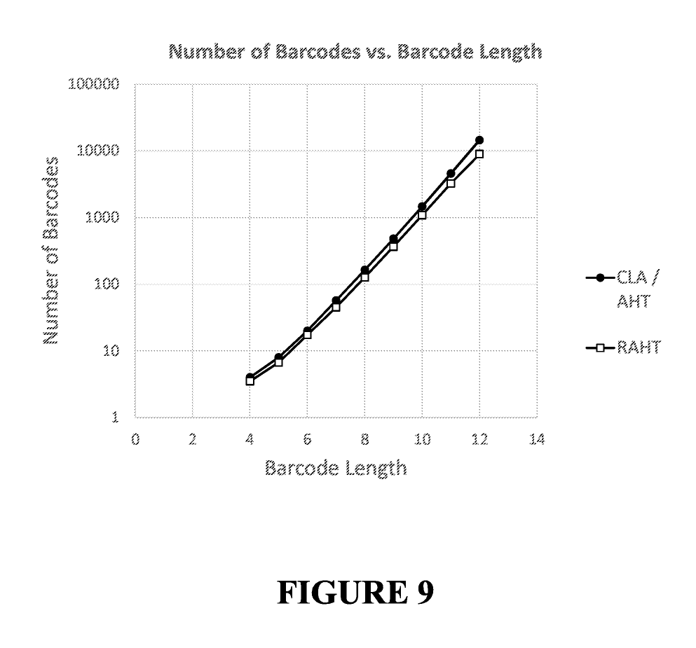

Two exemplary methods described in the present disclosure (i.e., AHT and RAHT) were utilized to generate sets of barcodes and the dependency of number of barcodes on barcode length found for each of the algorithms were plotted and compared to a known method (i.e., Conway's lexicode algorithm (CLA), see Conway J. et al. Information Theory, IEEE Transaction on. 32(3): 337-348), as shown in FIG. 9. The library edit distance (d) was set to 4 for all three algorithms. The running of RAHT was ceased until the rate of new barcodes being added to the library slowed to a level indicating the maximum number of barcodes (0.6 seconds/barcode for barcode length n≦10 and 1.2 seconds/barcode for barcode length n>10) was approached. For RAHT, the average number of barcodes after 10 runs for each n was calculated and taken. As the figure shows, unlike AHT, RAHT was non-deterministic, and the output of RAHT was different from that of CLA. The set of barcodes output by RAHT tended to be smaller than the set output by CLA for given n and d.

### Example 5: Methods and Execution Time

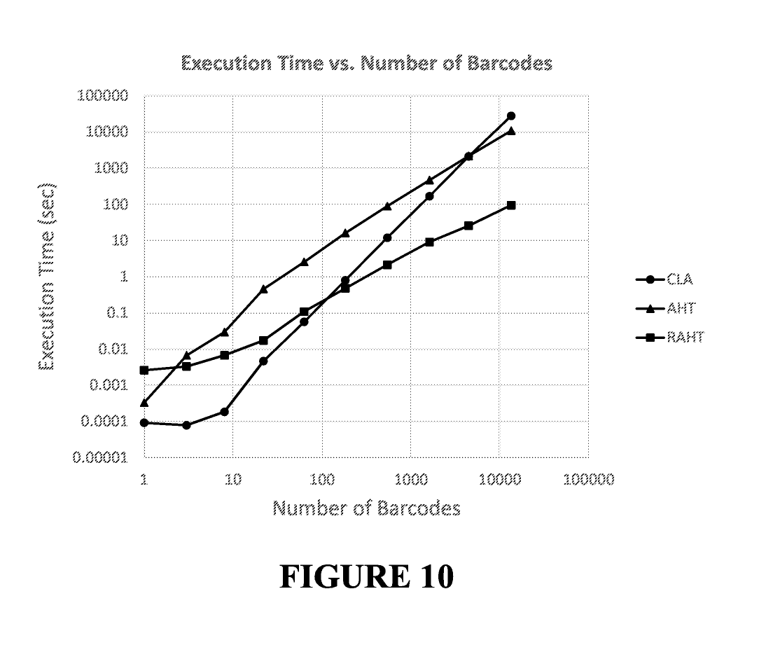

The execution time for methods CLA, AHT and RAHT to generate a certain number of barcodes with a specified library edit distance (d=4) were compared and shown in FIG. 10. For RAHT, a pre-determined set size (or number of barcodes contained in a set) m was set to half of the set size achieved with RAHT in example 4 (FIG. 9). For a given barcode length n, same set size m was chosen for all three methods. For RAHT, the average execution time after 10 runs for each n was taken. The methods were implemented in cython and run on an Intel Xeon X5675 (3.07 GHz) processor. With the use of a hash table, instead of computing edit distance between each candidate barcode and library barcode, the edit distance(s) only needed to be calculated when encountering a mutation (cⱼ) of candidate barcode, if hash value of the cⱼ was already present in the hash table. This greatly reduced the time required for sufficiently large n, as shown in FIG. 10. Moreover, given an appropriate choice of m, RAHT could be a worthwhile alternative to AHT since it ran faster than AHT within a certain range of set sizes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A set of barcodes comprising at least 1,500,000 barcodes with an edit distance of at least 2.

2. The set of barcodes of claim 1, comprising at least 5,000,000 barcodes.

3. The set of barcodes of claim 1, wherein the edit distance is at least 4.

4. The set of barcodes of claim 1, wherein each of the barcodes has a length of at least 15.

5. The set of barcodes of claim 1, wherein the set of barcodes has an error rate of 0.005% or less.

6. The set of barcodes of claim 1, wherein the barcodes comprise nucleic acid molecules.

7. The set of barcodes of claim 1, wherein additional information is associated with the barcodes.

8. The set of barcodes of claim 7, wherein the additional information comprises at least one of:
a. a complete nucleic acid sequence;
b. a source identifier; and
c. an information link.

9. The set of barcodes of claim 1, wherein the barcodes have a G:C content above a pre-determined threshold value.

10. The set of barcodes of claim 1, wherein the barcodes have a G:C content below a pre-determined threshold value.

11. The set of barcodes of claim 1, wherein the barcodes have less than four nucleotides in a row from the group consisting of A and T.

12. The set of barcodes of claim 1, wherein the barcodes have less than four nucleotides in a row from the group consisting of G and C.

13. The set of barcodes of claim 1, wherein the barcodes have a homopolymer run less than or equal to 4 nucleotides in length.

14. A method for generating a set of barcodes having a pre-determined library edit distance, comprising:
a. providing a set of library barcodes, wherein each of the library barcodes in the set of library barcodes comprises a library barcode index;
b. receiving a candidate barcode;
c. generating a first set of mutations of the candidate barcode;
d. converting the candidate barcode, each of the library barcodes and each of the first set of mutations of the candidate barcode into hash values using a hash function;
e. providing a creation hash table that relates each of the hash values of each of the library barcodes to its library barcode index;
f. comparing the hash values of the first set of mutations of the candidate barcode to the creation hash table, and if at least one of the hash values has been assigned to the library barcode index or indices in the creation hash table, then determining edit distances between the candidate barcode and the library barcode or the library barcodes indexed with the same hash value; and
g. adding the candidate barcode to the set of library barcodes if none of the determined edit distances from step (f) are less than the pre-determined library edit distance.

15. The method of claim 14, wherein the set of library barcodes is empty and the candidate barcode is added to the set of library barcodes without comparison.

16. The method of claim 14, wherein the set of library barcodes comprises at least one library barcode.

17. The method of claim 14, wherein the creation hash table is empty.

18. The method of claim 14, wherein each of the library barcodes has a length of at least 2.

19. The method of claim 14, wherein the candidate barcode has a length of at least 2.

20. The method of claim 14, wherein the library edit distance is at least 2.

21. The method of claim 14, further comprising: determining a creation hash table edit distance and a comparison edit distance according to the library edit distance by using the formula [the library edit distance=the creation hash table edit distance+the comparison edit distance+1].

22. The method of claim 21, wherein the first set of mutations of the candidate barcode is within the comparison edit distance of the candidate barcode.

23. The method of claim 21, further comprising: (i) generating one or more mutations of at least one of the library barcodes, wherein the mutations are within the creation hash table edit distance of the at least one of the library barcodes; (ii) converting the one or more mutations from (i) into hash values using the hash function; and (iii) relating the hash values from (ii) to the library barcode index of the at least one of the library barcode in the creation hash table.

24. The method of claim 21, further comprising:
h. assigning a new library barcode index to the added candidate barcode;
i. generating a second set of mutations of the added candidate barcode, wherein the second set of mutations is within the creation hash table edit distance of the added candidate barcode;
j. determining hash values of the second set of mutations of the added candidate barcode using the hash function; and
k. updating the creation hash table by pairing the new library barcode index with the hash values of the second set of mutations of the added candidate barcode.

25. The method of claim 14, further comprising receiving a set of candidate barcodes and selecting an individual candidate barcode from the set of candidate barcodes.

26. The method of claim 25, wherein the individual candidate barcode is selected in a random order.

27. The method of claim 25, further comprising selecting the next candidate barcode from the set of candidate barcodes if none of the hash values of the first set of mutations of the selected candidate barcode have been assigned to the library barcode index in the creation hash table.

28. The method of claim 27, further comprising keeping selecting the candidate barcode for comparison until the set of library barcodes comprises a pre-determined number of barcodes.

29. The method of claim 14, wherein the set of library barcodes comprises a plurality of nucleic acid molecules.

30. The method of claim 25, wherein the set of candidate barcodes comprises a plurality of nucleic acid molecules.

31. The method of claim 14, further comprising removing the candidate barcode with a G:C content above a pre-determined threshold value.

32. The method of claim 14, further comprising removing the candidate barcode with a G:C content below a pre-determined threshold value.

33. The method of claim 14, further comprising removing the candidate barcode capable of forming a hairpin structure.

34. The method of claim 14, further comprising removing the candidate barcode having a known restriction site.

35. The method of claim 14, further comprising removing the candidate barcode having a start codon.

36. The method of claim 14, further comprising removing the candidate barcode having forbidden sequences.

37. The method of claim 14, further comprising removing the candidate barcode having a homopolymer run greater than or equal to 2 nucleotides in length.

38. The method of claim 14, wherein the set of barcodes comprises at least 1,000,000 barcodes.

39. The method of claim 14, wherein the set of barcodes is generated in less than 250 hours.

40. The method of claim 14, wherein the set of barcodes is generated with a unit execution time of 1 s or less.

41. The method of claim 14, wherein the set of barcodes is used for nucleic acid sequencing.

42. A method for decoding a set of barcodes within a pre-determined resolution edit distance, the method comprising:
a. providing a set of library barcodes with the resolution edit distance, wherein each of the library barcodes in the set of library barcodes has a library barcode index;
b. selecting a candidate barcode from the set of barcodes;
c. converting the candidate barcode and each of the library barcodes into hash values using a hash function;
d. providing a decoding hash table that relates each of the hash values of the library barcodes to its library barcode index;
e. comparing the hash value of the candidate barcode to the decoding hash table, and if the hash value of the candidate barcode has already been assigned to the library barcode index or indices in the decoding hash table, then determining edit distances between the candidate barcode and the library barcode or the library barcodes indexed with the same hash value; and
f. matching the candidate barcode to the library barcode or library barcodes if the determined edit distances from step (e) are not greater than the resolution edit distance.

43. The method of claim 42, wherein the set of library barcodes is empty and the candidate barcode is added to the set of library barcode without comparison.

44. The method of claim 42, wherein the resolution edit distance is at least 1.

45. The method of claim 42, wherein each of the library barcodes has a length of at least 2.

46. The method of claim 42, wherein the candidate barcode has a length of at least 2.

47. The method of claim 42, wherein the candidate barcode has the same length as the library barcodes.

48. The method of claim 42, further comprising: (i) generating one or more mutations of at least one of the library barcodes, wherein the one or more mutations are within the resolution edit distance of the at least one of the library barcodes; (ii) converting each of the mutations of the at least one of the library barcodes into hash values using the hash function; and (iii) relating the hash values of the mutations of the at least one of the library barcodes to its library barcode index in the decoding hash table.

49. The method of claim 42, wherein the candidate barcode is selected from the set of barcodes in a random order.

50. The method of claim 42, further comprising marking the candidate barcode as “unresolvable” if all of the determined edit distances from step (e) are greater than the resolution edit distance.

51. The method of claim 42, further comprising repeating steps (b)-(f) until a pre-determined number of the candidate barcodes has been decoded.

52. The method of claim 42, wherein the set of library barcodes comprises nucleic acid molecules.

53. The method of claim 42, wherein the candidate barcode comprises nucleic acid molecule.

54. The method of claim 42, wherein the set of barcodes comprises at least 1,000,000 barcodes.

55. The method of claim 42, wherein the set of barcodes is decoded in less than 1,000 seconds.

56. The method of claim 42, wherein the set of barcodes is decoded with a unit execution time of 0.000001 s or less.

57. The method of claim 42, wherein the set of barcodes is decoded with a determination error rate of 1% or less.

58. A computer readable medium comprising codes that, upon execution by one or more computer processors, implements a method for generating a set of barcodes comprising at least 1,500,000 barcodes with a library edit distance of at least 2, in less than 24 hours.

59. The computer readable medium of claim 58, wherein the method comprises:
a. providing a set of library barcodes, wherein each of the library barcodes in the set of library barcodes comprises a library barcode index;
b. receiving a candidate barcode;
c. generating a first set of mutations of the candidate barcode;
d. converting the candidate barcode, each of the library barcodes and each of the first set of mutations of the candidate barcode into hash values using a hash function;
e. providing a creation hash table that relates each of the hash values of each of the library barcodes to its library barcode index;
f. comparing the hash values of the first set of mutations of the candidate barcode to the creation hash table, and if at least one of the hash values has been assigned to the library barcode index or indices in the creation hash table, then determining edit distances between the candidate barcode and the library barcode or the library barcodes indexed with the same hash value; and
g. adding the candidate barcode to the set of library barcodes if none of the determined edit distances from step (f) are less than the pre-determined library edit distance.

60. The computer readable medium of claim 59, wherein the method further comprises: determining a creation hash table edit distance and a comparison edit distance according to the library edit distance.

61. The computer readable medium of claim 60, wherein the method further comprises: (i) generating one or more mutations of at least one of the library barcodes, wherein the mutations are within the creation hash table edit distance of the at least one of the library barcodes; (ii) converting the one or more mutations from (i) into hash values using the hash function; and (iii) relating the hash values from (ii) to the library barcode index of the at least one of the library barcode in the creation hash table.

62. The computer readable medium of claim 60, wherein the method further comprises:
h. assigning a new library barcode index to the added candidate barcode;
i. generating a second set of mutations of the added candidate barcode, wherein the second set of mutations is within the creation hash table edit distance of the added candidate barcode;
j. determining hash values of the second set of mutations of the added candidate barcode using the hash function; and
k. updating the creation hash table by pairing the new library barcode index with the hash values of the second set of mutations of the added candidate barcode.

63. The computer readable medium of claim 59, wherein the method further comprises receiving a set of candidate barcodes and selecting an individual candidate barcode from the set of candidate barcodes.

64. The computer readable medium of claim 63, wherein the method further comprises selecting the next candidate barcode from the set of candidate barcodes if none of the hash values of the first set of mutations of the selected candidate barcode have been assigned to the library barcode index in the creation hash table.

65. The computer readable medium of claim 64, wherein the method further comprises keeping selecting the candidate barcode for comparison until the set of library barcodes comprises a pre-determined number of barcodes.

66. The computer readable medium of claim 58, wherein the set of barcodes is generated with a unit execution time of 1 s or less.

67. A computer readable medium comprising codes that, upon execution by one or more computer processors, implements a method for decoding a set of barcodes comprising at least 1,500,000 barcodes with a resolution edit distance of at least 1, in less than 1,000 s.

68. The computer readable medium of claim 67, wherein the method comprises:
a. providing a set of library barcodes with the resolution edit distance, wherein each of the library barcodes has a library barcode index;
b. selecting a candidate barcode from the set of barcodes;
c. converting the candidate barcode and each of the library barcodes into hash values using a hash function;
d. providing a decoding hash table that relates each of the hash values of each of the library barcodes to its barcode index;
e. comparing the hash value of the candidate barcode to the decoding hash table, and if the hash value of the candidate barcode has already been assigned to the library barcode index or indices in the decoding hash table, then determining an edit distance between the candidate barcode and the library barcode or the library barcodes indexed with the same hash value; and
f. matching the candidate barcode to the library barcode or library barcodes if the determined edit distance from step (e) is not greater than the resolution edit distance.

69. The computer readable medium of claim 68, wherein the method further comprises: (i) generating one or more mutations of at least one of the library barcodes; (ii) converting the one or more mutations of the at least one of the library barcodes into hash values using the hash function; and (iii) relating the hash values of the one or more mutations of the at least one of the library barcodes to its library barcode index in the decoding hash table.

70. The computer readable medium of claim 68, wherein the method further comprises marking the candidate barcode as “unresolvable” if all of the determined edit distances from step (e) are greater than the resolution edit distance.

71. The computer readable medium of claim 68, wherein the method further comprises repeating steps (b)-(f) until a pre-determined number of the candidate barcodes has been decoded.

72. The computer readable medium of claim 67, wherein the set of barcodes is decoded with a unit execution time of 0.000001 s or less.

73. The computer readable medium of claim 67, wherein the set of barcodes is decoded with a determination error rate of 1% or less.
